(19) United States
(12) Patent Application Publication
(10) Pub. No.: US 2006/0216696 A1
(43) Pub. Date: Sep. 28, 2006

(54) Rapid plague detection system

(76) Inventor: Jon D. Goguen, Holden, MA (US)

Correspondence Address: MIRICK, O'CONNELL, DEMALLIE & LOUGEE, 100 FRONT STREET, WORCESTER, MA, 01608, US

(21) Appl. No.: 10/568,862

(22) PCT Filed: Aug. 23, 2004
(86) PCT No.: PCT/US04/27256

§ 371 Date: Feb. 21, 2006

(51) Int. Cl. C12Q 1/70; G01N 33/554; C12M 1/34; G01N 33/569

(52) U.S. Cl. 435/5; 435/7.32; 435/287.2

(57) ABSTRACT

The present invention provides a system including a compact biological agent detector that determines the presence and amount of a biological agent by a specific immunoassay and furnishes a rapid and convenient readout of the immunoassay results. In another aspect, the invention provides a method of determining the presence and amount of a biological agent in a sample.

### CROSS REFERENCE TO RELATED APPLICATIONS

The present application claims the benefit of U.S. Provisional Application No. 60/496,731, filed Aug. 21, 2003. The entire content of the above application is incorporated herein by reference in entirety.

### BACKGROUND OF THE INVENTION

The U.S. public health system and primary health-care providers must now be prepared to address varied biological agents, including pathogens that are rarely seen in the United States. The Centers for Disease Control and Prevention (CDC) have classified the biological agents that might be used as weapons by the potential effects on the population and the public health system. High-priority agents (Category A) agents include organisms that pose a risk to national security because they can be easily disseminated or transmitted person-to-person; cause high mortality, with potential for major public health impact; might cause public panic and social disruption; and require special action for public health preparedness. A Category A agents include anthrax (Bacillus anthracis), botulism (Clostridium botulinum toxin), plague (Yersinia pestis), smallpox (Variola major), tularemia (Francisella tularensis) and viral hemorrhagic fevers (filoviruses [e.g., Ebola, Marburg] and arenaviruses [e.g., Lassa, Machupo]) (Table 1). The second highest priority agents (Category B agents) include those that are moderately easy to disseminate; cause moderate morbidity and low mortality; and require specific enhancements of CDC's diagnostic capacity and enhanced disease surveillance (Table 1). The third highest priority agents (Category C agents) include emerging pathogens that could be engineered for mass dissemination in the future because of availability; ease of production and dissemination; and potential for high morbidity and mortality and major health impact (Table 1). See generally, CDC Strategic Planning Workgroup, Biological and Chemical Terrorism: Strategic Plan for Preparedness and Response, Centers for Disease Control and Prevention Morbidity and Mortality Weekly Report, Recommendations and Reports, Apr. 21, 2000/Vol. 49/No. RR-4, available at http://www.cdc.gov/mmwr/PDF/rr/rr4904.pdf.

Yersinia pestis, the causative agent of plague, is one of the most serious of the limited number of Category A agents that could cause disease and death in sufficient numbers to cripple a city or region (Inglesby, T. V., et al., Plague as a biological weapon: medical and public health management. Working Group on Civilian Biodefense. JAMA. 2000 May 3; 283(17):2281-90). Given the availability of Y. pestis around the world, capacity for its mass production and aerosol dissemination, difficulty in preventing such activities, high fatality rate of pneumonic plague, and potential for secondary spread of cases during an epidemic, the potential use of plague as a biological weapon is of great concern. (Inglesby, 2000).

TABLE 1
Critical biological agents

Category A agents include
variola major (smallpox);
Bacillus anthracis (anthrax);
Yersinia pestis (plague);
Clostridium botulinum toxin (botulism);
Francisella tularensis (tularaemia);
Filoviruses: Ebola hemorrhagic fever, Marburg hemorrhagic fever; and
Arenaviruses: Lassa (Lassa fever), Junin (Argentine hemorrhagic fever)
and related viruses
Category B agents include
Coxiella burnetti (Q fever);
Brucella species (brucellosis);
Burkholderia mallei (glanders);
alphaviruses (Venezuelan encephalomyelitis, eastern & western equine
encephalomyelitis);
ricin toxin from Ricinus communis (castor beans);
epsilon toxin of Clostridium perfringens; and
Staphylococcus enterotoxin B.
A subset of Category B agents includes pathogens that are food- or
waterborne.
These pathogens include but are not limited to
Salmonella species,
Shigella dysenteriae,
Escherichia coli O157:H7,
Vibrio cholerae, and
Cryptosporidium parvum.
Category C agents include
nipah virus,
hantaviruses,
tickborne hemorrhagic fever viruses,
tickborne encephalitis viruses,
yellow fever, and
multidrug-resistant tuberculosis.

The first recorded plague pandemic began in AD 541 in Egypt and swept across Europe with population losses of between 50% and 60% in North Africa, Europe, and central and southern Asia. The second plague pandemic, also known as the Black Death or great pestilence, began in 1346 and eventually killed 20 to 30 million people in Europe, one third of the European population. Plague spread slowly and inexorably from village to village by infected rats and humans or more quickly from country to country by ships. The pandemic lasted more than 130 years and had major political, cultural, and religious ramifications. The third pandemic began in China in 1855, spread to all inhabited continents, and ultimately killed more than 12 million people in India and China alone.

Plague is currently present on every major inhabited continent, except Australia. In 1899, it was introduced into the United States via San Francisco. Currently plague, along with yellow fever and cholera, are the only three diseases that have worldwide quarantine sanctions. Small outbreaks of plague continue to occur throughout the world. Advances in living conditions, public health, and antibiotic therapy make future pandemics improbable. However, plague outbreaks following use of a biological weapon are a plausible threat.

In World War II, a secret branch of the Japanese army, Unit 731, is reported to have dropped plague-infected fleas over populated areas of China, thereby causing outbreaks of plague. In the ensuing years, the biological weapons programs of the United States and the Soviet Union developed techniques to aerosolize plague directly, eliminating dependence on the unpredictable flea vector. In 1970, the World Health Organization (WHO) reported that, in a worst-case scenario, if 50 kg of Y. pestis were released as an aerosol over a city of 5 million, pneumonic plague could occur in as many as 150,000 persons, 36,000 of whom would be expected to die. The plague bacilli would remain viable as an aerosol for 1 hour for a distance of up to 10 km. Significant numbers of city inhabitants might attempt to flee, further spreading the disease (Inglesby, 2000).

Natural transmission of the organism Yersinia pestis is through the bite of infected fleas. Of the approximately 1,500 species of fleas, only 30 or so are proven vectors, with oriental rat flea being the most competent. The average life span of an uninfected flea is about 6 weeks, though some may live as long as a year under certain conditions. A plague-infected flea lives only about 17 days because it dies from starvation and dehydration.

Human plague most commonly occurs when plague-infected fleas bite humans who then develop bubonic plague. As a prelude to human epidemics, rats frequently die in large numbers, precipitating the movement of the flea population from its natural rat reservoir to humans. Although most persons infected by this route develop bubonic plague, a small minority will develop sepsis with no bubo (acutely swollen tender lymph node), a form of plague termed primary septicemic plague. Neither bubonic nor septicemic plague spreads directly from person to person. A small percentage of patients with bubonic or septicemic plague develop secondary pneumonic plague and can then spread the disease by respiratory droplet. Persons contracting the disease by this route develop primary pneumonic plague. Pneumonic plague is the only form of plague that can be spread person-to-person.

As noted above, plague used as a biological weapon would most likely be dispersed as an aerosol of Y. pestis. Symptoms would begin to appear 1 to 6 days after exposure, and those exposed would die soon after onset of symptoms (Inglesby, 2000). Primary pneumonic plague resulting from the inhalation of plague bacilli occurs rarely in the United States. Reports of 2 recent cases of primary pneumonic plague, contracted after handling cats with pneumonic plague, reveal that both patients had pneumonic symptoms as well as prominent gastrointestinal symptoms including nausea, vomiting, abdominal pain, and diarrhea. Diagnosis and treatment were delayed more than 24 hours after symptom onset in both patients, both of whom died (Inglesby, 2000).

The fatality rate of patients with pneumonic plague is extremely high when treatment (parenteral antibiotics) is delayed more than 24 hours after symptom onset (Inglesby, 2000). In addition, postexposure prophylactic antibiotic treatment should be started promptly for those who have been exposed to the infected patient.

However, there are no widely available rapid diagnostic tests for plague. Tests that would be used to confirm a suspected diagnosis—antigen detection, IgM enzyme immunoassay, immunostaining, and polymerase chain reaction—are available only at some state health departments, the CDC, and military laboratories. The routinely used passive hemagglutination antibody detection assay is typically only of retrospective value since several days to weeks usually pass after disease onset before antibodies develop. A Gram stain of sputum or blood may reveal gram-negative bacilli or coccobacilli. Microscopic examination after Wright, Giemsa, or Wayson stain will often show bipolar staining of the bacterium, and direct fluorescent antibody testing, if available, may be positive. Cultures of sputum, blood, or lymph node aspirate should demonstrate growth approximately 24 to 48 hours after inoculation (Inglesby, 2000). However, none of these available diagnostic tests provides convenient rapid detection of the pathogen at the point of care.

Recently more rapid “dipstick” diagnostic tests have been developed based on antibodies to the F1 antigen and compared to ELISA diagnostic tests (Chanteau, S., et al., Early diagnosis of bubonic plague using F1 antigen capture ELISA assay and rapid immunogold dipstick, Int. J. Med. Microbiol., 290:279-283 (2000); Chanteau, S., et al., Development and testing of a rapid diagnostic test for bubonic and pneumonic plague. Lancet, 361:211-216 (2003)). However, the F1 antigen is not detectable in Y. pestis grown at 25 degrees Celsius as opposed to 37 degrees Celsius (Phillps, A. P., et al., Identification of encapsulated and non-encapsulated Yersinia pestis by immunofluorescence tests using polyclonal and monoclonal antibodies, Epidemiol. Infect. 101:59-73 (1988)). F1-negative strains have been isolated from natural sources, and F1 has been shown not to be a required virulence factor (Davis, K. J., et al., Pathology of experimental pneumonic plague produced by fraction 1-positive and fraction 1-negative Yersinia pestis in African green monkeys (Cercopithecus aethiops), Arch Pathol Lab Med. 120(2):156-63 (1996)). A need for vaccines and diagnostic assays that are not solely based on the F1 antigen has been identified (Davis et al., 1996).

### SUMMARY OF THE INVENTION

The present invention provides a system including a compact biological agent detector that determines the presence and amount of a biological agent by a specific immunoassay and detection by imaging fluorophores excited by an optical evanescent field and furnishes a rapid and convenient readout of the immunoassay results. Biological agents from a specimen, such as pathogenic microorganisms and biological toxins, are suspended in an analysis solution contained in a sample chamber, the analysis solution comprising a first antibody specific to the biological agent comprising target analyte, wherein the first antibody is affixed to a substrate which undergoes relative movement with respect to the analysis solution. In preferred embodiments the target analyte comprises an epitope of the biological agent that is recognized by the first antibody. The analysis solution further comprises a first complex of the first antibody conjugated to a first fluorophore. The biological agent detector further comprises an excitation light source and at least one photodetector.

In preferred embodiments, the analysis solution further comprises a second antibody that is conjugated to a second fluorophore that is distinguished from the first fluorophore by spectral characteristics. The second antibody is specific for an antigen that is irrelevant to the target analyte, and is used to control for non-specific binding. In preferred embodiments, the sample chamber has an optical waveguide. In some embodiments, the optical waveguide forms a side, top or bottom of the sample chamber. In other embodiments, the optical waveguide is movably disposed within the sample chamber. In some embodiments, the optical waveguide is an optical waveguide array.

In one embodiment, the biological agent detector comprises a sample chamber having an optical waveguide and enclosing an analysis solution; a first antibody fixed to a movable substrate, wherein the first antibody is specific to the biological agent that is the target analyte; a conjugate of the first antibody and a first fluorophore; an excitation light source and a photodetector.

In preferred embodiments, the biological agent detector further comprises a movable substrate actuator. In preferred embodiments, the biological agent detector further comprises an image analyzer. Typically, the photodetector is an imaging emitted light detector, and preferably is a CCD detector. In preferred embodiments, the analysis solution is a buffered aqueous solution. In preferred embodiments, the analysis solution further comprises a second antibody that is conjugated to a second fluorophore, wherein the second fluorophore is distinguished from the first fluorophore by spectral characteristics.

Typically the first antibody is selected from the group consisting of a polyclonal antibody specific for the biological agent; a monoclonal antibody specific for the biological agent; an antibody fragment specific for the biological agent; a recombinant antibody specific for the biological agent and mixtures thereof. Typically the second antibody is selected from the group consisting of a polyclonal antibody specific for an irrelevant protein; a monoclonal antibody specific for an irrelevant protein; an antibody fragment specific for an irrelevant protein; a recombinant antibody specific for an irrelevant protein; and mixtures thereof.

In preferred embodiments, the first antibody is affixed to a substrate that undergoes relative movement with respect to the analysis solution.

In a preferred embodiment, the substrate is movable, and preferably is a paramagnetic bead and the movable substrate actuator is a permanent magnet. As used herein “paramagnetic” means that the beads respond to a magnetic field, but are not magnets themselves and retain no residual magnetism after removal of the magnet. In another embodiment, the movable substrate is a movable array of optical waveguides or a convoluted optical waveguide and the movable substrate actuator is chosen from mechanical and electromechanical devices including levers, slides, gears, solenoids, electromagnets and combinations thereof.

In addition to binding to the first antibody fixed to the movable substrate, the biological agent binds to molecules of the first antibody that are conjugated to a first fluorophore to form a complex labeled by the first fluorophore. In preferred embodiments in which the movable substrate is a paramagnetic bead, this fluorophore-labeled complex is moved into the optical evanescence field of a planar optical waveguide by the influence of a magnetic field on the paramagnetic beads. In preferred embodiments, the planar optical waveguide is illuminated with light of a wavelength that excites the first fluorophore, causing excitation of the fluorophores in the fluorophore-labeled complexes that are present in the optical evanescence field of the planar optical waveguide. In preferred embodiments, the planar optical waveguide forms the top, bottom or a side of the sample chamber. Preferably, the side of the sample chamber that comprises the planar optical waveguide is imaged onto a photodetector. Most preferably, the photodetector is a charge-coupled device (CCD) detector, a CMOS detector or other flat panel imaging sensor.

In embodiments in which the first antibody is fixed to a waveguide array that comprises the movable substrate, binding of the biological agent to the affixed first antibody molecules brings the biological agent into the optical evanescence field of the waveguide array. Binding of molecules of the first antibody that are conjugated to a first fluorophore to the bound biological agents forms a complex labeled by the first fluorophore within the optical evanescence field. Illumination of the waveguide array with light of a wavelength that excites the first fluorophore causes excitation of the fluorophores in the fluorophore-labeled complexes that are present in the optical evanescence field of the waveguide guide array. The waveguide array is moved into the object plane of the optical system and imaged onto the photodetector.

In some embodiments incorporating a second antibody, the second fluorophore that is conjugated to the second antibody is characterized by an excitation spectrum that overlaps the excitation spectrum of the first fluorophore and an emission spectrum that is distinguishable from the emission spectrum of the first fluorophore. The light emitted by each fluorophore excited in the optical evanescence field is imaged onto a CCD detector, a CMOS detector or other flat panel imaging sensor. In other embodiments incorporating a second antibody, the second fluorophore that is conjugated to the second antibody is characterized by an excitation spectrum with minimal overlap with the excitation spectrum of the first fluorophore, such that excitation wavelengths can be chosen that respectively excite substantially only the first fluorophore and substantially only the second fluorophore, thereby providing a spectral distinction. Such combinations of fluorophores may be further distinguished by different emission spectra.

The CCD detector provides an output signal that is representative of the position, intensity and wavelength of the light emitted by the fluorophores in the optical evanescence field of the optical waveguide. The wavelength of the emitted light can be determined by comparing images obtained using optical band pass filters chosen to select for specific fluorophores. Alternatively, where the fluorophores are distinguished by excitation at different wavelengths, the emission of the respective fluorophores can be sampled during excitation with the corresponding range of excitation wavelengths.

In preferred embodiments, the output signal of the photodetector, preferably a CCD detector, is the input to an image analyzer. The image analyzer can be contained in the same unit as the biological agent detector or can be in a different unit. In one preferred embodiment, the image analyzer is in the same unit as the biological agent detector.

In one embodiment, the image analyzer compares the number of pixels signaling the presence of a wavelength or band of wavelengths emitted by the first fluorophore and the amplitude of those signals, the first fluorophore emission signal, to the number of pixels signaling the presence of a wavelength or band of wavelengths emitted by the second fluorophore the amplitude of those signals, the second fluorophore emission signal, to provide a measure of antibody binding that is specific to the presence and amount of the biological agent that is the target analyte. In a preferred embodiment, the first fluorophore emission signal and the second fluorophore emission signal detected by each pixel are compared ratiometrically. The image analyzer displays the result of the comparison of the first fluorophore emission signal and the second fluorophore emission signal as a numeric value that is representative of the presence and amount of the biological agent that is the target analyte. In a preferred embodiment, the image analyzer transmits the numeric value that is representative of the presence and amount of the biological agent to a central computer. In preferred embodiments, the image analyzer comprises a display, and an image of the first fluorophore emission, an image of the second fluorophore emission or a transform thereof is displayed.

The present invention also provides a method of determining the presence and amount of a biological agent in a sample comprising placing the sample in a sample chamber having an optical waveguide; contacting the sample with an analysis solution comprising a buffer and reagents, the reagents comprising a first antibody affixed to a movable substrate, a conjugate of the first antibody and a first fluorophore, wherein the first antibody is specific to the biological agent that is the target analyte; reacting the sample with the reagents to form a complex of the target analyte with the first antibody labeled by the first fluorophore; moving the complex into the optical evanescence field of a optical waveguide; irradiating the optical evanescence field with an excitation light source; imaging the light emitted by excited fluorophores on a photodetector; producing a signal representative of the light emitted by excited fluorophores; processing the signal to produce a value representative of the presence and amount of the biological agent based on the specific binding of the first antibody; and reporting the value to determine the presence and amount of the biological agent in a sample. In preferred embodiments, the analysis solution further comprises a second antibody that is conjugated to a second fluorophore that is distinguishable from the first fluorophore by spectral characteristics, wherein the second antibody is specific for an antigen that is irrelevant to the biological agent. In preferred embodiments, the method further includes the step of using an actuator to sweep the movable substrate through the analysis solution.

The biological agent detector is useful for detecting biological agents such as Yersinia pestis (plague), variola major (smallpox), Bacillus anthracis (anthrax), Francisella tularensis (tularaemia), filoviruses such as Ebola hemorrhagic fever and Marburg hemorrhagic fever, arenaviruses such as Lassa (Lassa fever), Junin (Argentine hemorrhagic fever) and related viruses, alphaviruses (Venezuelan encephalomyelitis, eastern & western equine encephalomyelitis), Coxiella burnetti (Q fever), Brucella species (brucellosis), Burkholderia mallei (glanders), Salmonella species, Shigella dysenteriae, Escherichia coli O157:H7, Vibrio cholerae, Cryptosporidium parvum, nipah virus, hantaviruses, tickborne hemorrhagic fever viruses, tickborne encephalitis viruses, yellow fever, multidrug-resistant tuberculosis, Clostridium botulinum toxin (botulism), ricin toxin from Ricinus communis (castor beans), epsilon toxin of Clostridium perfringens, Staphylococcus enterotoxin B and mixtures thereof.

### BRIEF DESCRIPTION OF THE DRAWINGS

FIG. 1 is a schematic diagram of an embodiment of a biological agent detector.

FIG. 2 is a schematic diagram of another embodiment of a biological agent detector.

FIG. 3A is a schematic diagram of another embodiment of a biological agent detector.

FIG. 3B is a schematic diagram of another embodiment of a biological agent detection system employing a plurality of magnets.

FIG. 3C is a schematic diagram of another embodiment of a biological agent detection system employing a plurality of permanent magnets operating in conjunction with electromagnets.

FIG. 3D is a schematic diagram of another embodiment of a biological agent detection system employing magnets located on either side of the chamber.

FIG. 3E is a schematic diagram of an embodiment of a handheld embodiment of the biological agent detection system.

FIG. 4A is a schematic diagram of the excitation optics of an embodiment of a biological agent detector comprising a movable waveguide array as seen from the plane of the imaged side of the sample chamber 300.

FIG. 4B is a schematic diagram of the moveable waveguide array as seen perpendicular to the plane of FIG. 4A. Arrow 286 indicates the direction of translocation of the waveguide array.

FIG. 4C is a schematic diagram of another embodiment comprising a convoluted moveable waveguide array as seen from the plane of the imaged side of the sample chamber, 300 showing a substantially flattened coiled waveguide array 284 and exit beam trap 290.

FIG. 4D is a schematic diagram of another embodiment comprising a convoluted moveable waveguide array as seen from the plane of the imaged side of the sample chamber, 300 showing a substantially flattened coiled waveguide array 284 and photodetector 550.

FIG. 4E is a schematic diagram of an embodiment of the biological agent detector comprising a moveable waveguide array 282 that is translocated through sample chamber 300 in a direction shown by arrow 286 towards the imaged side of the sample chamber 300.

FIG. 4F is a schematic diagram of the optics an embodiment of a biological agent detector comprising imaging lens system 440, two charge coupled devices 500 and 510 with separate optics comprising an additional dichroic mirror 422, a band pass filter 432, and a short wavelength cut-off filter 434.

FIG. 5 is a schematic diagram of the relationship of the evanescent illumination zone of an optical waveguide, including analyte components and reagent components.

FIG. 6 is a graphical representation of the normalized fluorescence emission spectra of a family of 0.4 μm diameter fluorescent particles (TransFluoSpheres™ available from Molecular Probes, Eugene, Oreg.) excited by 488 nm light (wavelength indicated by the arrow in each spectrum). FIG. 6A shows the emission spectrum of a fluorophore emitting a peak wavelength of 560 nm. FIG. 6B is the spectrum of a fluorophore emitting at a peak wavelength of 605 nm. FIG. 6C is the emission spectrum of a fluorophore emitting at a peak wavelength of 645 nm. FIG. 6D is the emission spectrum of a fluorophore emitting at a peak wavelength of 685 nm. FIG. 6E is the emission spectrum of a fluorophore emitting at a peak wavelength of 720 nm.

FIG. 7 is a schematic diagram of an embodiment of an image analyzer.

FIG. 8 is a schematic diagram of a biological agent detection system.

FIG. 9 is a flowchart of an embodiment of a biological agent detection method.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

As used herein, the term “biological agents” includes pathogenic microorganisms and biological toxins. Pathogenic microorganisms include Yersinia pestis (plague), variola major (smallpox), Bacillus anthracis (anthrax), Francisella tularensis (tularaemia), filoviruses such as Ebola hemorrhagic fever and Marburg hemorrhagic fever, arenaviruses such as Lassa (Lassa fever), Junin (Argentine hemorrhagic fever) and related viruses, alphaviruses (Venezuelan encephalomyelitis, eastern & western equine encephalomyelitis), Coxiella burnetti (Q fever), Brucella species (brucellosis), Burkholderia mallei (glanders), Salmonella species, Shigella dysenteriae, Escherichia coli O157:H7, Vibrio cholerae, Cryptosporidium parvum, nipah virus, hantaviruses, tickborne hemorrhagic fever viruses, tickborne encephalitis viruses, yellow fever, and multidrug-resistant tuberculosis. Biological toxins include Clostridium botulinum toxin (botulism), ricin toxin from Ricinus communis (castor beans), epsilon toxin of Clostridium perfringens; and Staphylococcus enterotoxin B. In general, “microorganism” refers to any noncellular or unicellular (including colonial) organism, most of which are to small to be seen with the unaided eye, such as bacteria (including cyanobacteria), lichens, microfungi, protozoa, rickettsiae, virinos, viroids and viruses.

The present invention provides a system including a compact biological agent detector that determines the present and amount of a biological agent by a specific immunoassay and flushes a rapid and convenient readout of the immunoassay results. In a preferred embodiment, the system is compact enough to be hand-held. Biological agents, such as pathogenic microorganisms or toxins, from a specimen are suspended in an aqueous analysis solution comprising a buffer and reagents. The specimen can be a sample of biological fluids such as blood, plasma, urine, exudate, mucous or sputum. In an embodiment in which the biological agent is Yersinia pestis, the specimen is a throat swab. In another embodiment, the specimen is a vaginal swab and the biological agent is Chlamydia or Mycoplasma.

The reagents comprise a first antibody specific to the biological agent that is the target analyte fixed to a movable substrate, molecules of the first antibody conjugated to a first fluorophore having a first emission spectrum, and a control second antibody that is conjugated to a second fluorophore having an emission spectrum distinguishable from that of the first fluorophore. The second antibody is specific for an antigen that is irrelevant to the target analyte, and is used to control for non-specific binding.

In a preferred embodiment, the movable substrate is a paramagnetic bead. As used herein “paramagnetic” means that the beads respond to a magnetic field, but are not magnets themselves and retain no residual magnetism after removal of the magnet. Paramagnetic beads can be substantially suspended in a medium, such as a fluid, such that the beads have a likelihood of becoming associated with one or more biological agents proximate to the beads. It is known in the art that suspended beads can be directed to a location using fixed or variable magnetic fields. For example, if beads are suspended in a solution in a sample chamber, the beads can be drawn out of suspension and made to collect along an inner surface of the sample chamber using a magnetic field.

In another embodiment, the antibody-coated movable substrate is an array of optical waveguides. Movement, directly or indirectly by the influence of a magnetic field on the paramagnetic beads, moves the complexes of antibodies, fluorophores, substrate and biological agent into the evanescent illumination zone of an optical waveguide.

In a preferred embodiment, the fluorescent labels are commercially available, encapsulated fluorophores that have substantial Stokes shifts enabling detection of multiple analytes from a single sample. In preferred embodiments, the use of multiple fluorophores having distinguishable emission spectra provides the means for simultaneous detection of multiple biological agents. In some embodiments, the encapsulated fluorophores are quantum dot-fluorophores.

In a preferred embodiment, antibodies are directed towards biological toxin or to proteins expressed on the surface of a pathogenic microorganism, avoiding the need for lysis and allowing for a rapid assay format. During a short incubation period (˜15 min), specific bacteria within the sample are bound by the biological agent specific fluorescent-labeled antibody that for any given biological agent contains a unique fluorophore signature, and bound by the antibody on the paramagnetic beads. Non-specific binding by both the biological agent-specific antibodies and control antibodies to other bacteria and non-bacterial components within the sample occurs during this time. Following incubation, the paramagnetic beads and captured biological agent are drawn against one wall of the sample container by placing it within a strong magnetic field. This wall of the sample container is transparent, and serves as a waveguide into which is injected light capable of exciting multiple fluorophores simultaneously. The optical waveguide provides excitation energy restricted to the optical evanescence field, a thin layer (about 0.4 to about 1.2 μm) of the sample immediately adjacent to the waveguide surface. Despite waveguiding of the exciting radiation within the waveguide resulting from total internal reflection, the optical electromagnetic field extends beyond the waveguide surface by evanescent field leakage, and allows optical energy to be captured by objects in close proximity (of the order of one wavelength) to the surface. This excitation technique greatly reduces background fluorescence from antibodies in the bulk of the sample volume and eliminates the need for extensive wash steps for background reduction, further simplifying the assay format. Light emitted by excited fluorophores through the wall of the sample chamber serving as the excitation waveguide is imaged onto a CCD detector and converted into an electrical signal representative of the distribution and intensity of the light emitted.

The use of the control antibody and a ratiometric measurement controls for non-specific antibody binding. Ratios exceeding an experimentally determined value are considered indicative of the presence of a specific biological agent. Standard curves allow the determination of the amount of biological agent present.

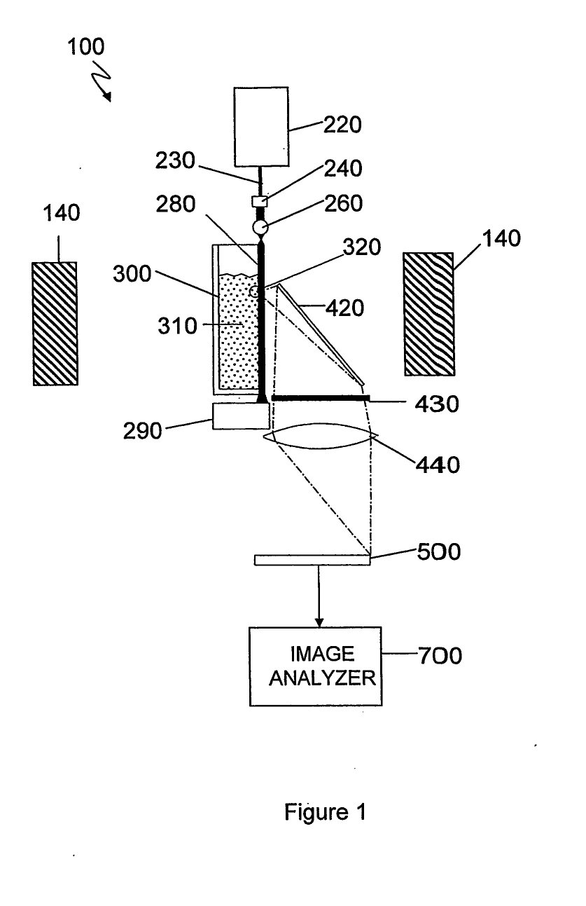

FIG. 1 is a schematic diagram of an embodiment of a biological agent detector. Light that excites label fluorophores is provided by the excitation light source 220, conditioning optics 240, waveguide coupling optics 260, optical waveguide 280 and is absorbed at the exit beam trap 290. In a preferred embodiment, the optical waveguide 280 is a planar optical waveguide that forms the imaged side of a sample chamber 300. The sample chamber 300 contains an analyte and reagent mixture 310 when in use. In preferred embodiments, the sample chamber 300 is sterile and disposable and can be fitted with a closure. A suitable sample container is a disposable cuvette. One suitable disposable cuvette is the Eppendorf Uvette® (Brinkmann, Westbury, N.Y.). In one embodiment, packaged sterile sample chambers pre-filled with buffer and reagents are prepared for each biological agent or mixture of biological agents and identified by a label that includes a bar code. In one embodiment, the bar code identifier on the sample chamber is automatically read as the sample chamber is inserted into the biological agent detector and appropriate measurement and analysis programs are selected.

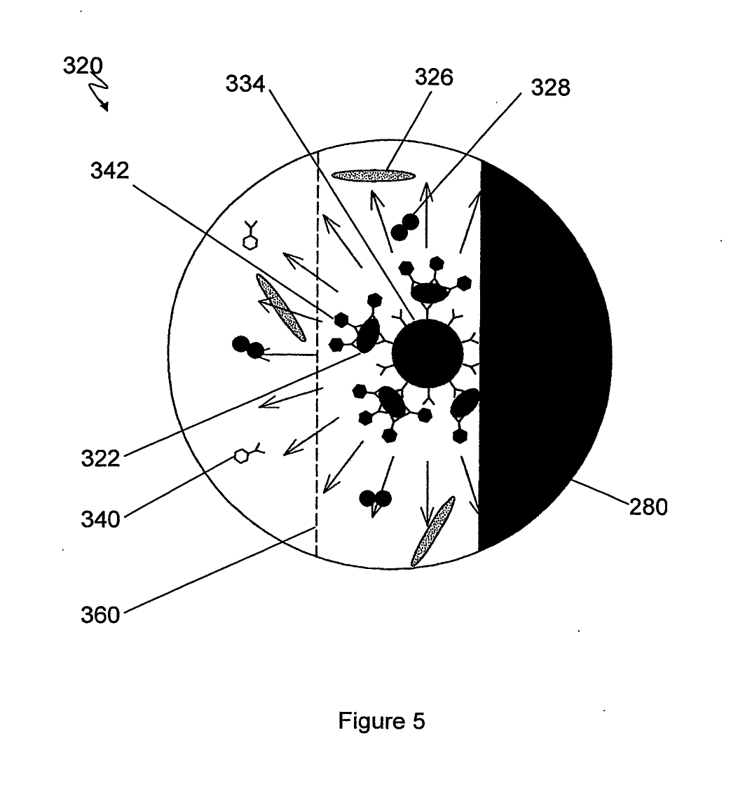

Paramagnetic beads and the associated reagents are swept through the volume of the analyte and reagent mixture 310 and moved into the optical evanescence zone adjacent to the optical waveguide 280 by the action of magnet 140. Details of the analyte-reagent interactions 320 in and near the optical evanescence zone are diagramed in FIG. 5. Light emitted by excited fluorophores in the optical evanescence zone adjacent to the optical waveguide 320 is imaged on a charge coupled device (CCD) detector 500 by means of mirror 420, filter 430 and imaging lens system 440. The imaging lens system comprises one or more optical elements that are refractive (dioptric) or reflective (catadioptric). Suitable reflective elements include low f number off-axis mirrors. Mirror 420 can be a dichroic mirror that acts to reflect towards the CCD detector 500 the wavelengths that are emitted by the excited fluorophores and transmitting other wavelengths. The output of the CCD detector 500 is processed by the image analyzer 700.

In preferred embodiments, filter 430 is a filter carrier, such as a slide or a filter wheel, that holds several filters of different band pass transmission that are matched to the emission spectra of the corresponding fluorophores. For example, if the emission peak wavelength of the first fluorophore is about 645 nm, a suitable corresponding bandwidth filter 430 transmits light of about 630 nm to about 660 nm, or about 635 nm to about 655 nm. Similarly, the emission peak wavelength of the second fluorophore is 560 nm, the corresponding bandwidth filter 430 transmits light from about 550 nm to about 580 nm. In general, the bandwidth of each filter 430 is chosen to minimize the contribution of the emission by another fluorophore. A filter carrier, if used, is moved from position to position manually or electromechanically, e.g., using a solenoid, DC motor or stepping motor.

Typically the magnet 140 exerts magnetic force on the paramagnetic beads and the attached antibody-analyte complexes, forcing the beads bearing the fluorophore-labeled complexes to accumulate on the right side of the sample cell 300 shown in FIG. 1. In a preferred embodiment, the approximate area of the accumulated beads is about 0.5 to 1 cm². Suitable paramagnetic beads can be obtained from several commercial sources, such as Bangs Laboratories (Fisher, Ind.).

When light of the excitation wavelength is applied to the optical waveguide, the evanescent optical field of excitation light the fluorophores of antibody-analyte complexes adjacent to the imaged side of the sample chamber results in fluorescent emission. Fluorescence originates only from the fluorophore labeled antibodies-analyte complexes immediately adjacent to the imaged side of the sample cell. Other sample material does not fluoresce because the evanescent fluorescence-excitation field does not penetrate the sample cell more than approximately one wavelength of excitation light.

The excitation light source 220 includes at least one light source selected from a number of suitable light sources including xenon arc lamp, xenon flash lamp, light emitting diodes (LEDs), laser diodes and lasers. Several exemplary suitable light sources are listed in Table 2, below.

TABLE 2
Excitation Light Sources

| | | | Nominal | |
|---|---|---|---|---|
| | | | intensity or | |
| Light Source | Wavelength | | power | |
| Xenon arc lamp, xenon flash lamp | Broad, | | Up to 175 | W |
| | About 14% of | | | |
| | power in the 400-500 | | nM | |
| | band | | | |
| Sapphire 488-20 CW blue laser (Coherent, Inc, Santa | 488 ± 2 | nM | 20 | mW |
| Clara, CA) | | | | |
| Sapphire 460-10 CW blue laser (Coherent, Inc, Santa | 460 ± 2 | nM | 10 | mW |
| Clara, CA) | | | | |
| Compass 405-25 diode laser (Coherent, Inc, Santa | 405 | nM | 25 | mW |
| Clara, CA) | | | | |
| LS-450 Blue LED Pulsed Light Source (Ocean Optics, | 470 | nM | 50 | μW |
| Inc., Dunedin, FL) | | | | |
| Lepton II Diode Laser (Micro Laser Systems, Garden | 440 | nM | 3 | mW |
| Grove, CA) | | | | |
| Blue (InGaN) diode (Kingbright Corp. City of Industry, | 460 | nM | 1400 | mcd |
| CA) | | | | |
| IQ2 diode laser (Power Technology, Inc., Little Rock, | 405 ± 10 | nM | 20 | mW |
| AR) | | | | |
| PPM25 diode laser (Power Technology, Inc., Little | 405 ± 10 | nM | 25 | mW |
| Rock, AR) | | | | |
| Blue (InGaN) diode (Fairchild Semiconductor, South | 465 | nM | 650 | mcd |
| Portland, ME) | | | | |
| Lasiris green laser (StockerYale Canada, Montreal, CA) | 532 | nM | 10 | mW |
| Green diode laser 532-20-E (B & W Tek, Newark, DE) | 532 | nM | 20 | mW |
| Sanyo Laser diode DL4038-026 (Thorlabs, Inc., | 635 | nM | 20 | mW |
| Newton, NJ) | | | | |
| Hitachi Laser diode HL6320G (Thorlabs, Inc., Newton, | 635 | nM | 10 | mW |
| NJ) | | | | |

In one preferred embodiment, the excitation light source 200 emits light in a single band of wavelengths, e.g., centered on 488 nm, that is matched to the excitation spectra of all fluorophores used, and the fluorophores can be spectrally distinguished on the basis of their emission spectra. In other embodiments, the fluorophores can be spectrally distinguished on the basis of their excitation spectra, and at least two bands of excitation wavelengths are provided by the excitation light source. In some such embodiments, the excitation light source comprises at least two separate light sources, each selected independently from the group consisting of xenon arc lamp, xenon flash lamp, light emitting diode (LED), laser diode and laser, that provide light of separate bands of excitation wavelengths. In one embodiment, the excitation light source comprises at least two lasers or laser diodes having differing emission bandwidths centered at 488 nm, 532 nm or 635 nm. In other embodiments, the separate bands of excitation wavelengths are provided by a single light source, such as a xenon lamp, by using dichroic mirrors and/or band-pass blocking filters.

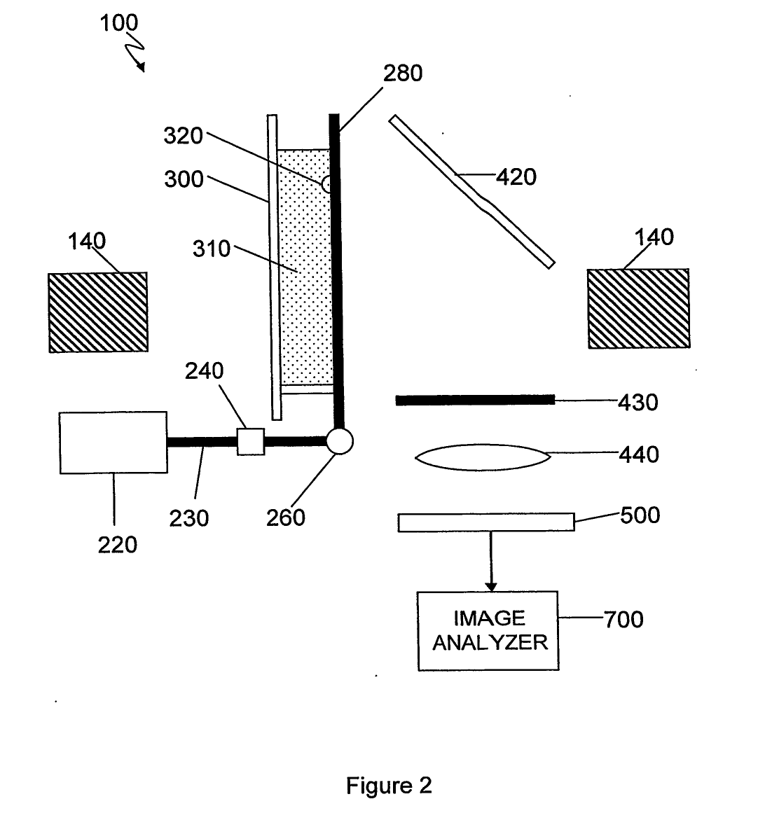

FIG. 2 is a schematic diagram of another embodiment of a biological agent detector. The excitation light that is provided by the excitation light source 220 is conveyed by an excitation optical waveguide 230, conditioning optics 240 and waveguide coupling optics 260 to the bottom of a planar optical waveguide 280 that serves as a side of the sample chamber. The sample chamber 300 contains an analyte and reagent mixture 310 when in use. In preferred embodiments, the sample chamber 300 is sterile and disposable and can be fitted with a closure. A suitable sample container is a disposable cuvette. One suitable disposable cuvette is the Eppendorf Uvette® (Brinkmann, Westbury, N.Y.). Paramagnetic bead and the associated reagents are moved into the optical evanescence zone adjacent to the optical waveguide 280 by the action of magnet 140. Light emitted by excited fluorophores in the optical evanescence zone adjacent to the optical waveguide 280 is imaged on a charge coupled device (CCD) detector 500 by means of mirror 420, filter 430 and imaging lens system 440. As in the embodiment of FIG. 1, filter 430 can be a multiple bandpass filter array, such as a filter wheel, with the transmission characteristics of each bandpass filter chosen to match the emission peak wavelength of a corresponding fluorophore. In one embodiment, mirror 420 is a dichroic mirror that acts to reflect towards the CCD detector 500 the wavelengths that are emitted by the excited fluorophores but passing wavelengths of the excitation light. The output of the CCD detector 500 is processed by the image analyzer 700.

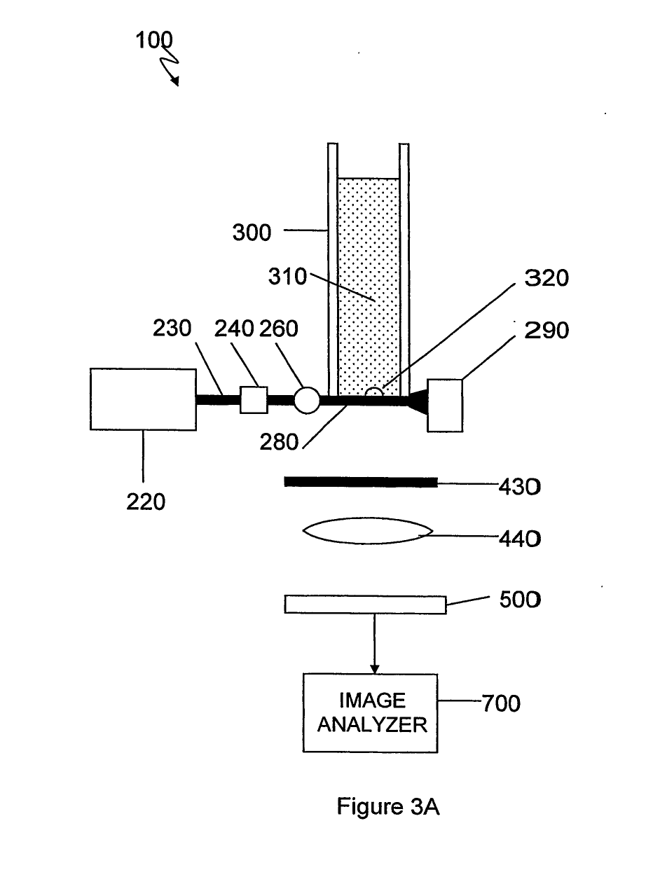

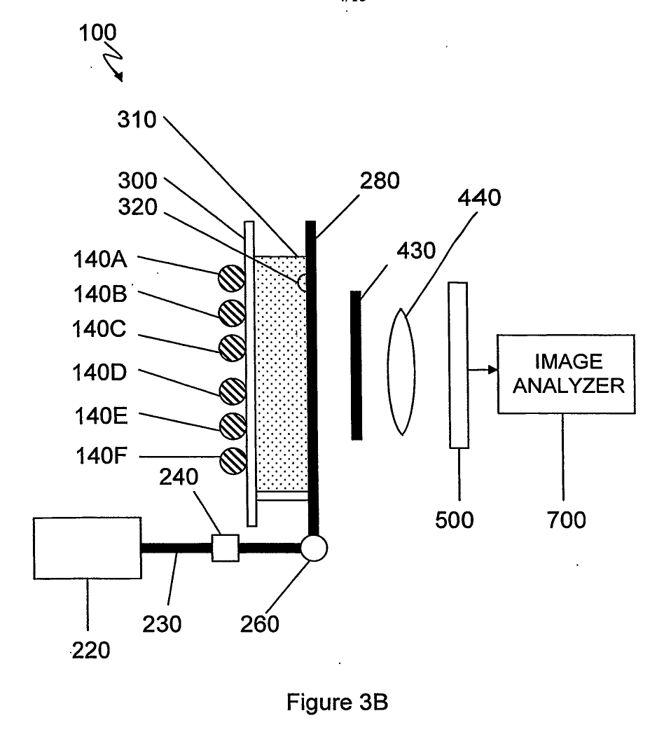

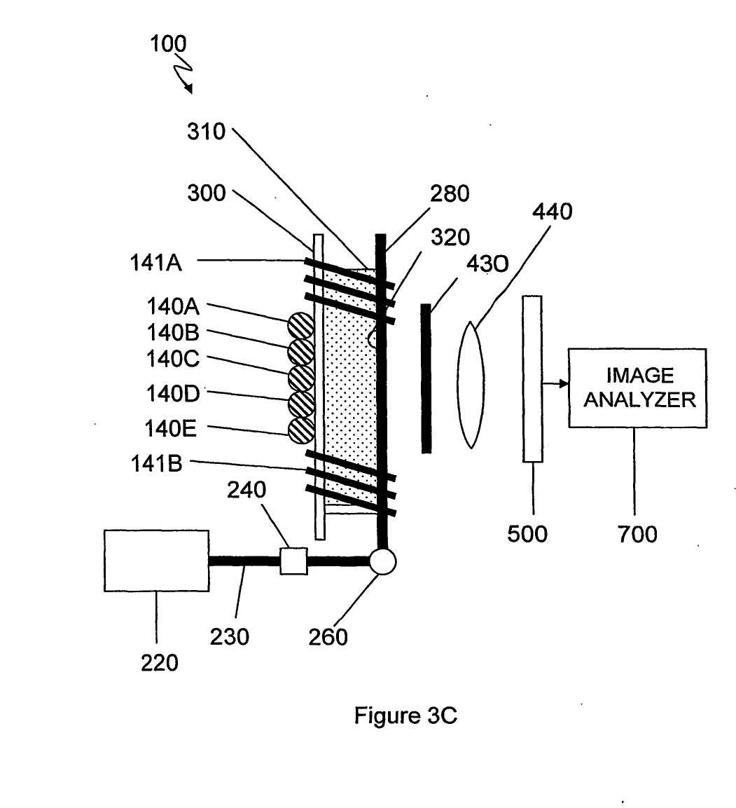

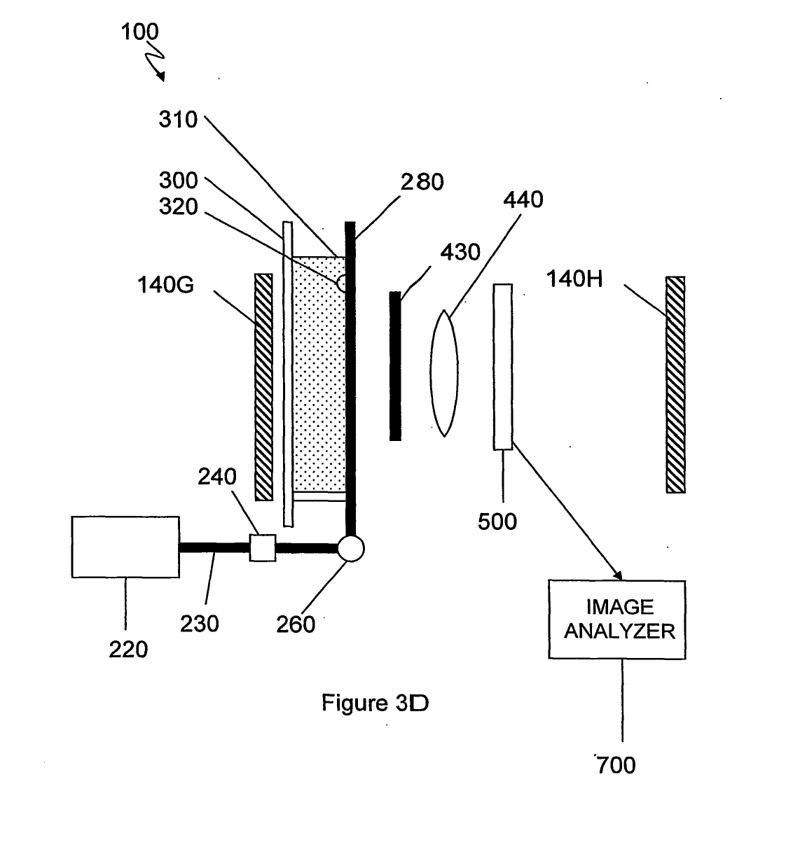

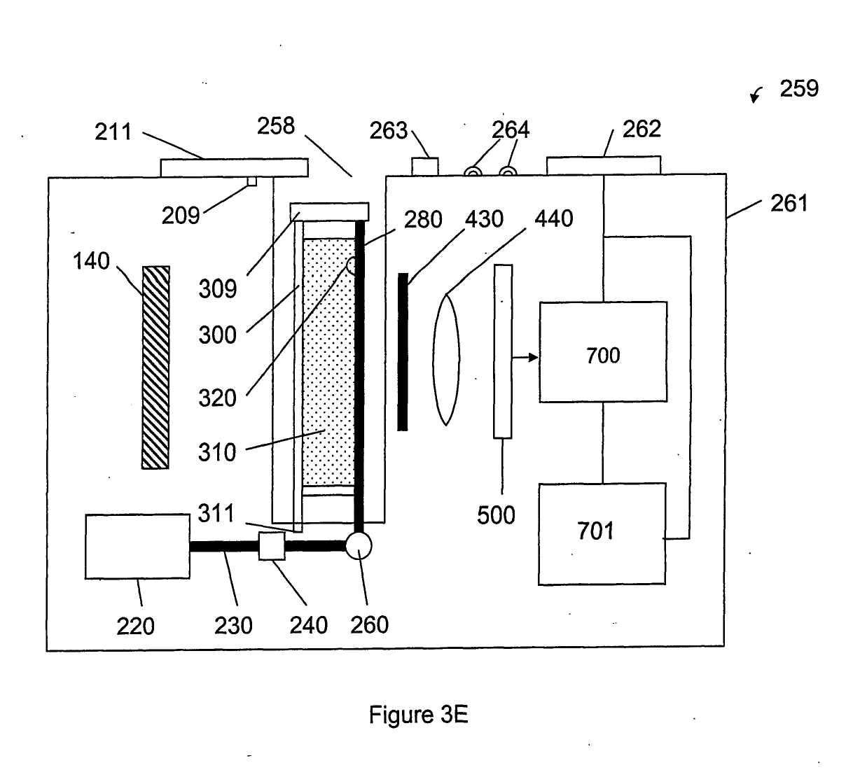

FIG. 3A is a schematic diagram of another embodiment of biological agent detector. Excitation light is provided by the excitation light source 220 and conveyed by an excitation optical waveguide 230, conditioning optics 240 and waveguide coupling optics 260 to a planar optical waveguide 280 that forms that bottom of the sample chamber 300. In this embodiment, the paramagnetic beads are large enough so that captured labeled antibody analyte complexes are removed from the volume of the analyte reagent mixture 310 by the action of gravity. The fluorophore-labeled antibody analyte complexes attached to the paramagnetic beads on the bottom of sample chamber are imaged onto the CCD detector 500 by light passing through filter 430 and imaging lens system 440.

FIG. 3B is a schematic diagram of another embodiment of the biological agent detector. The configuration of FIG. 3B is similar to that of FIG. 3A, differing in the position of the planar optical waveguide 280 along with other imaging components with respect to the imaged side of chamber 300. The configuration of FIG. 3B is useful when the analyte and reagent mixture 310 contains contaminating particles having sufficient density to sink to the bottom of the chamber 300. Contaminating particles may include, for example, sediment, organisms or organic matter other than those to be detected using the specific antibody. The embodiment of FIG. 3B also includes a plurality of magnets 140A-140F instead of the single magnet 140 of FIG. 3A. Magnets 140A-F are disposed along the non-imaged side of chamber 300 in a manner facilitating propagation of paramagnetic beads and fluorophores toward the imaged side of chamber 300. Magnets 140A-F may be permanent magnets, electromagnets or a combination of permanent magnets and electromagnets.

FIG. 3C is a schematic diagram of an embodiment of the biological agent detector employing both permanent magnets 140A-E and electromagnets 141A and 141B. The electromagnets may be driven in a steady state or in a gated state while operating in conjunction with permanent magnets to cause paramagnetic beads and fluorophores to move towards planar optical waveguide 280.

FIG. 3D is a schematic diagram of an embodiment of the biological agent detector employing two magnets 140G and 140H located on either side of reaction chamber 300. Magnets 140G and H may be constructed as permanent magnets, electromagnets or as a combination of permanent and electromagnets. Magnets 140G and 140H are arranged so that the magnetic field between them causes paramagnetic beads with affixed biological agents and fluorophores to be displaced towards planar optical waveguide 280.

The embodiments illustrated in FIGS. 3A-3D include magnets that are located within a detection apparatus in an orientation whereby the magnetic flux associated with the magnet(s) causes the paramagnetic beads with associated biological agents and fluorophores to move towards planar optical waveguide 280 and to eventually come to rest within the optical evanescence field in substantial contact with planar optical waveguide 280. The magnets used in embodiments of FIGS. 3A-D may further be shaped or conformed to produce a magnetic field having a gradient that causes paramagnetic beads to move toward planar optical waveguide 280.

FIG. 3E is a schematic diagram of an exemplary embodiment of a handheld unit 259 that can be used to detect biological agents in accordance with methods disclosed herein. In FIG. 3E, a handheld unit 259 employs a case 261 for holding the elements making up the biological agent detector. The handheld unit 259 contains an opening 258 formed in the case 261 which is adapted to receive a chamber 300 containing an analyte and reagent mixture 310 to be analyzed. Chamber 300 has a sealably mounted lid 309 attached thereto to prevent spillage of the analyte and reagent mixture 310. The lid 309 preferably makes a positive seal with the chamber 300 to avoid contamination of the handheld unit 259. Chamber 300 may have planar optical waveguide 280 associated therewith, or planar optical waveguide 280 may be mounted in handheld unit 259. When chamber 300 is fully inserted into case 261, planar optical waveguide 280 is removably coupled to waveguide coupling optics 260, and a light-tight cover 211 is positioned to isolate the sample chamber 300 from light sources external to the unit. In preferred embodiments, the sample chamber 300 is encoded on a non-imaged surface with a symbol code, preferably bar-coded, providing information about the reagents contained, biological agents (analytes) to be identified and sample identity. The code is read by a code reader 312 and the information read is provided to processor 701.

A user may initiate the detection and analysis procedure by pushing an “analyze” button 263 which causes handheld unit 259 to analyze the contents of chamber 300 using image analyzer 700. Alternatively, the detection and analysis procedure can be initiated automatically, incorporating the output of sensors 209 and 311 that detect the closure of the cover 211 and the seating of the sample chamber 300, respectively. Image analyzer 700 is coupled to display 262 which is used to present analysis results to the user and processor 701 which implements the detection and analysis procedure. Processor 701 can have integral memory storage or separate memory storage. Handheld unit 259 may also include one or more status LED's 264 for informing a user about the operation of unit 259. For example, a red LED may be used to inform the user that the detection and analysis procedure was not completed properly, while a green LED may be used to indicate error-free completion. In certain preferred embodiments, the unit includes appropriate sensors and instructions to detect common errors, such as a failure to seat the sample chamber properly and provide messages. Processor 701 can have integral memory storage or separate memory storage. Handheld unit 259 may be sized to comfortably fit a user's hand and operated while wearing protective clothing or may be configured for installation in a vehicle, backpack, or as a module within another piece of analysis or detection equipment. Handheld unit 259 may be powered using internal batteries, solar cells, or by connection to an external power source such as a vehicle's electrical system or to a standard wall outlet providing alternating current (AC).

In other embodiments, the optical waveguide of the biological agent detector can be an array of at least one optical waveguide that is swept through the volume of the sample solution 310. In such embodiments, the movable waveguide array is moved by an substrate actuator that is chosen from mechanical and electromechanical devices including levers, slides, gears, solenoids, electromagnets and combinations thereof.

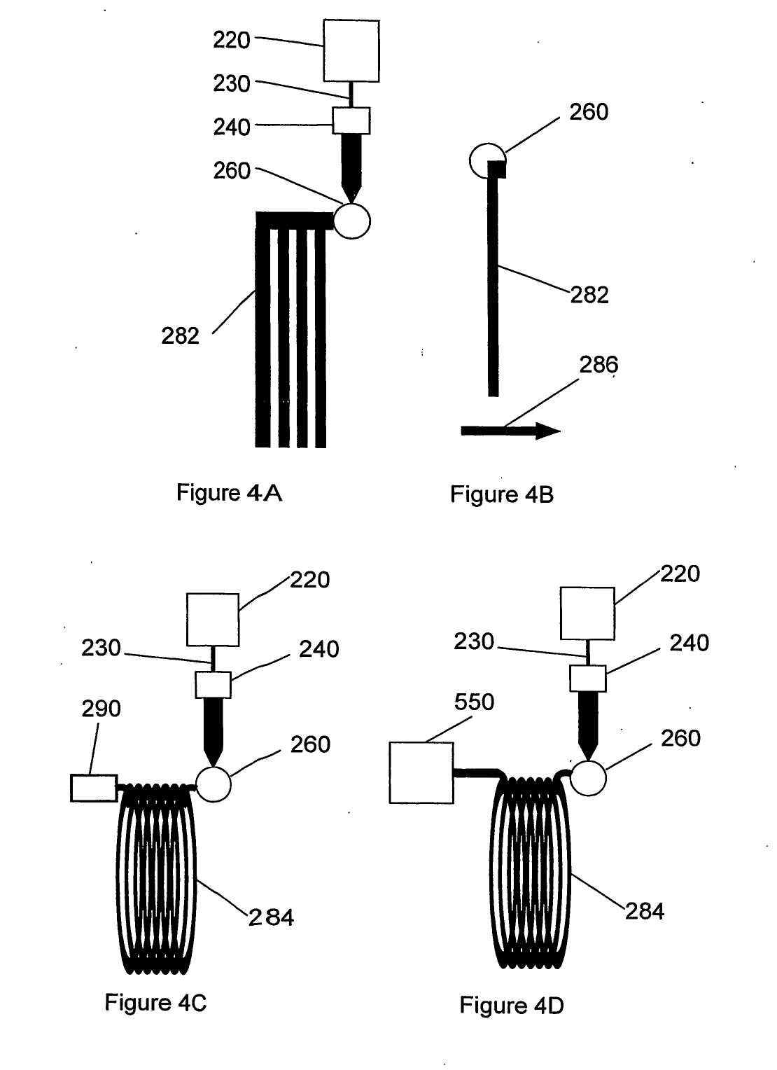

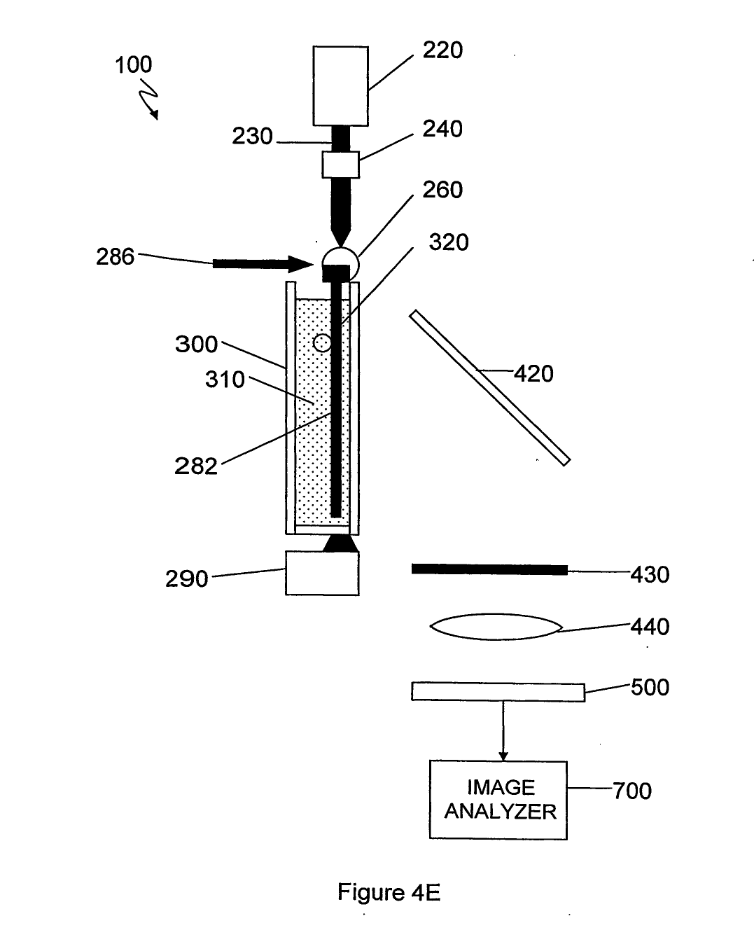

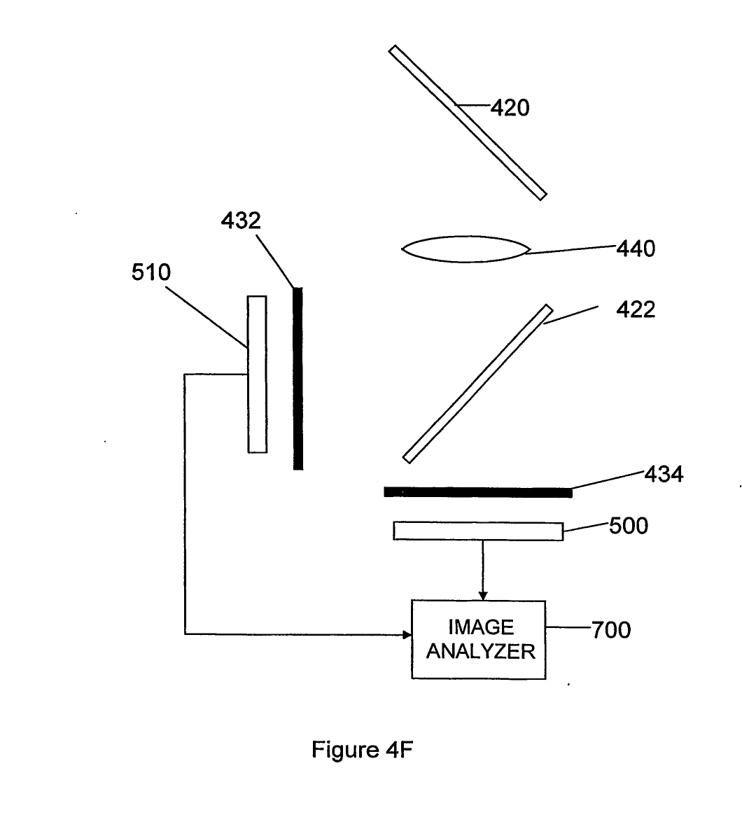

FIG. 4A is a schematic diagram of one embodiment of a waveguide array that is swept through the volume of the analyte and reagent mixture 310. As with other embodiments the excitation light source 220, light source waveguide 230, conditioning optics 240, and waveguide coupling optics 260 are present. In preferred embodiments, the geometry of the waveguide array is chosen to accommodate the dimensions of the sample chamber 300, maximize surface area and maximize the fraction of the waveguide array falling within the field of the object plane of the imaging optical system. In this embodiment the waveguide array 282 is in the form of a brush with several pendant smaller waveguides attached to the coupling optics. This array is shown in FIG. 4A in a plane parallel to the imaged side of the sample chamber that approximates the field of the object plane of the imaging optical system. In FIG. 4B the waveguide array 282 and coupling optics 270 are shown in a plane perpendicular to that of FIG. 4A. FIG. 4C is a schematic diagram of another embodiment comprising a convoluted moveable waveguide array shown the plane of the field of the object plane of the imaging optical system showing a substantially flattened coiled waveguide array 284 and exit beam trap 290.

FIG. 4D is a schematic diagram of another embodiment comprising a convoluted moveable waveguide array as seen from the plane of the imaged side of the sample chamber, 300 showing a substantially flattened coiled waveguide array 284 and photodetector 550. In such embodiments, the amount of bound analyte can be measured by a wavelength-dependent reduction in transmission through the waveguide. In such embodiments, the antibodies can be labeled with absorptive dyes.

In the embodiments depicted in FIGS. 4A, 4B and 4C, the waveguide array 282 or 284 is coated with an antibody that is specific for the analyte. Thus, when the waveguide array is swept through the volume of the sample, the antibody coding on the waveguide array collects analyte and complexes formed by labeled antibody and the analyte. When the waveguide array is in position adjacent to imaged side of the sample chamber, the light from light source 220 excites the fluorophores within the evanescence zone around the fibers of the waveguide array, producing a light pattern that is imaged on the photodetector 500.

FIG. 4E is a schematic diagram of an embodiment of the biological agent detector comprising a moveable array of waveguides 282 that is translated through sample chamber 300 in a direction shown by arrow 286 towards the imaged side of the sample chamber 300. The waveguide array 282 comes to rest in the object plane of the imaging optical system 420, 430 and 440.

FIG. 4F is a schematic diagram of the optics of an embodiment of a biological agent detector comprising an imaging lens system 440, two charge coupled devices 500 and 510 with separate optics comprising an additional dichroic mirror 422, a band pass filter 432 and a short wavelength cut off filter 434.

FIG. 5 is a schematic diagram of interactions between analytes and reagents within the light 360 of the optical evanescence field of an optical waveguide 280. Shown are target analyte 322, in this case the biological agent Yersina pestis, bound to both antibody specific for this biological agent that is linked to a paramagnetic bead 334 and excited fluorophore-labeled antibody specific for this biological agent 342. Note that fluorophore-labeled antibody molecules 340 that are outside of the optical evanescence field are not excited. Non-target analytes 326 and 328 are also shown.

Antibody Reagents

Antibody reagents can be prepared using any number of techniques known in the art. Suitable techniques are discussed briefly below.

The conventional direct fluorescence assay uses an antibody specific for the capsular polypeptide of Yersinia pestis known as Fraction 1 antigen or, more simply, as F1. In one embodiment, anti-F1 antibody serves as the basis for specific pathogen detection. However, since Yersinia pestis mutants lacking F1 can remain highly virulent for primates via the aerosol route, in another embodiment, at least one antibody specific for a necessary Yersinia pestis virulence determinant is used. Suitable alternative antigens include V antigen, YpkA, YopH, YopM, YopB, YopD, YopN, YopE, YopK, Pla, pH 6 antigen and purified LPS (Benner, G. E., et al., Immune response to Yersinia outer proteins and other Yersinia pestis antigens after experimental plague infection in mice, Infect Immun. 1999 April; 67(4):1922-8). Preferred alternative antigens include V antigen, YopH, YopM, YopD and Pla. In preferred embodiments, an antibody to the Pla antigen is used.

The antibody may be polyclonal or monoclonal. Polyclonal antibodies have significant advantages for initial development, including rapidity of production and specificity for multiple epitopes, ensuring strong immunofluorescent staining and antigen capture. Monoclonal antibodies are adaptable to large-scale production; preferred embodiments include at least one monoclonal antibody specific for Yersinia pestis. Because polyclonal preparations cannot be readily reproduced for large-scale production, another embodiment uses a cocktail of at least four monoclonal antibodies.

A single chain Fv (“scFv” or “sFv”) polypeptide is a covalently linked V_{H}:V_{L} heterodimer which may be expressed from a nucleic acid including V_{H}- and V_{L}-encoding sequences either joined directly or joined by a peptide-encoding linker. Huston, et al. Proc. Nat. Acad. Sci. USA, 85: 5879-5883 (1988). A number of structures for converting the naturally aggregated, but chemically separated, light and heavy polypeptide chains from an antibody V region into an scFv molecule which folds into a three dimensional structure substantially similar to the structure of an antigen-binding site. See, e.g. U.S. Pat. Nos. 6,512,097, 5,091,513 and 5,132,405 and 4,956,778.

In one class of embodiments, recombinant design methods can be used to develop suitable chemical structures (linkers) for converting two naturally associated—but chemically separate—heavy and light polypeptide chains from an antibody variable region into a sFv molecule which folds into a three-dimensional structure that is substantially similar to native antibody structure. Design criteria include determination of the appropriate length to span the distance between the C-terminal of one chain and the N-terminal of the other, wherein the linker is generally formed from small hydrophilic amino acid residues that do not tend to coil or form secondary structures. Such methods have been described in the art. See, e.g., U.S. Pat. Nos. 5,091,513 and 5,132,405 to Huston et al.; and U.S. Pat. No. 4,946,778 to Ladner et al.

In this regard, the first general step of linker design involves identification of plausible sites to be linked. Appropriate linkage sites on each of the V_{H} and V_{L} polypeptide domains include those which result in the minimum loss of residues from the polypeptide domains, and which necessitate a linker comprising a minimum number of residues consistent with the need for molecule stability. A pair of sites defines a “gap” to be linked. Linkers connecting the C-terminus of one domain to the N-terminus of the next generally comprise hydrophilic amino acids which assume an unstructured configuration in physiological solutions and preferably are free of residues having large side groups which might interfere with proper folding of the V_{H} and V_{L} chains. Thus, suitable linkers under the invention generally comprise polypeptide chains of alternating sets of glycine and serine residues, and may include glutamic acid and lysine residues inserted to enhance solubility. Nucleotide sequences encoding such linker moieties can be readily provided using various oligonucleotide synthesis techniques known in the art.

The phrase “specifically binds to a protein” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to a protein under such conditions may require an antibody that is selected for its specificity for a particular protein. For example, antibodies can be raised to the F1 protein that bind F1 and not to other proteins present in a sample. A variety of immunoassay formats may be used to select antibodies specifically immunoreactive with a particular protein. For example, solid-phase ELISA immunoassays are routinely used to select monoclonal antibodies specifically immunoreactive with a protein.

A chimeric molecule is a molecule in which two or more molecules that exist separately in their native state are joined together to form a single molecule having the desired functionality of all of its constituent molecules. While the chimeric molecule may be prepared by covalently linking two molecules each synthesized separately, one of skill in the art will appreciate that where the chimeric molecule is a fusion protein, the chimera may be prepared de novo as a single “joined” molecule.

The term “conservative substitution” is used in reference to proteins or peptides to reflect amino acid substitutions that do not substantially alter the activity (specificity or binding affinity) of the molecule. Typically conservative amino acid substitutions involve substitution one amino acid for another amino acid with similar chemical properties (e.g. charge or hydrophobicity), examples are found in Table 3, below.

TABLE 3
Conservative Amino Acid Substitutions

The following six groups each contain amino acids that are typical
conservative substitutions for one another:
Alanine (A), Serine (S), Threonine (T);
Aspartic acid (D), Glutamic acid (E);
Asparagine (N), Glutamine (Q);
Arginine (R), Lysine (K);
Isoleucine (I), Leucine (L), Methionine (M), Valine (V);
Phenylalanine A), Tyrosine (Y), Tryptophan (W).

Preparation of Polyclonal Plague Antibodies and Non-Specific Control Antibodies

In one embodiment, the fluorescence assay utilizes an antibody specific for the capsular polypeptide of Y. pestis known as Fraction 1 antigen or, more simply, as F1. Because Y. pestis mutants lacking F1 can remain highly virulent for primates via the aerosol route, inclusion of a second antibody specific for a non-dispensible Y. pestis virulence determinant can be isolated using substractive screening (see below). Use of such an antibody is useful for detecting virulent strains that have been engineered not to display epitopes recognized by the anti-F1 antibodies. Polyclonal antibodies have significant advantages, including rapidity of production and specificity for multiple epitopes, ensuring strong immunoFluorescent staining and antigen capture.

Production of Polyclonal Anti-F1.

Polyclonal anti-F1 is produced in rabbits via immunization with highly purified F1 capsular polypeptide isolated from Y. pestis strain KIM. This strain has been used extensively in studies of Y. pestis virulence and is one of two Y. pestis strains for which the sequence of the genome has been completed. For isolation of F1, the avirulent derivative KIM5 is used to reduce biohazard concerns. KIM5 lacks the pCDI plasmid encoding the type III secretion system which is required for virulence by all routes of infection, and also carries a 100 kilobase chromosomal deletion that removes genes critical for iron acquisition during infection. As a result, this strain is essentially avirulent.

F1 is isolated from the supernatant of cultures grown in Heart Infusion Broth supplemented with 0.2% xylose at 37° C. A well-established method, previously used to produce F1 for use in vaccine testing, can be employed (Andrews G P, Heath D G, Anderson G W Jr, Welkos S L, Friedlander A M. Fraction 1 capsular antigen (F1) purification from Yersinia pestis CO92 and from an Escherichia coli recombinant strain and efficacy against lethal plague challenge. Infect Immun. 1996 June; 64(6): 2180-7). Briefly, F1 is precipitated from the culture supernatant with 30% ammonium sulfate, dialyzed against phosphate-buffered saline (PBS), and further purified by preparative gel filtration. The later step takes advantage of the strong tendency of F1 to form high-molecular-weight aggregates at neutral pH. This causes F1 to migrate at the exclusion limit of Superdex 200 Hiload resin (1.3×106 Da), while migration of contaminating proteins and a minimal amount of F1 are retarded. Several-milligram quantities of F1 can be readily processed in a column of modest size (1 liter bed volume). Following this gel filtration step, endotoxin is removed from the purified material via affinity chromatography with polymixin B. Purity of the F1 preparation is confirmed by examination of silver-stained SDS-PAGE gels. A yield of 15 milligrams per liter of culture is typical. Two to three liters of culture thus yields sufficient antigen for subsequent steps, including affinity purification of the antibody.

Rabbits are immunized with purified F1 protein adsorbed to aluminum hydroxide adjuvant by standard methods. Similar preparations are known to produce high antibody titers in vaccine trials. Immunization and serum recovery are performed on a fee for service basis by various vendors, such as Strategic Biosolutions (Newark, Del.). For example, four rabbits are immunized using an accelerated protocol that provides antibody 50 days after immunization. Anti-F1 titers following test bleeds are determined by ELISA using pre-bleed serum as a negative control.

Purification of F1-Specific Antibody.

Rabbit anitsera prepared against purified F1 is not suitable for use in later experiments without further purification since it contains antibodies that react with bacteria present in the normal human flora recovered by throat swab. This is due to the previous exposure of the rabbits to closely related bacteria. F1-specific antibody is purified from the high-titer rabbit serum in two steps. First, IgGs is purified via affinity chromatography on Protein A sepharose via standard methods. Anti-F1 IgG is then isolated from the purified IgG fraction via chromatography on a column containing highly purified F1 immobilized on the activated immunoaffinity support Affi-Gel 15 (BioRad), an N-hydroxylsuccinimide ester of derivitized cross-linked aragose beads. The 15 atom spacer carrying the reactive succinimide in Affi-Gel 15 contains a cationic charge, which makes it particularly well suited for coupling of acidic proteins at neutral pH, an important consideration for coupling F1, which has a calculated isoelectric point of 4.35. Coupling efficiency is monitored by assaying the amount of unbound protein remaining after the coupling reaction is complete.

F1 exists primarily in oligomeric form. Initial affinity columns are prepared without special treatment of F1. The oligomeric state of F1 on the column can be disrupted during elution of antibody by the conditions used for elution of antibody (3 M NaSCN), leading to loss of antigen from the column and contamination of the antibody with this released antigen. F1 oligomers have been reported to be rather stable, requiring treatment at 100° C. in SDS or exposure to 7 M urea for disruption (Miller J, Williamson E D, Lakey J H, Pearce M J, Jones S M, Titball R W. Macromolecular organisation of recombinant Yersinia pestis F1 antigen and the effect of structure on immunogenicity. FEMS Immunol Med Microbiol. 1998 July; 21(3):213-21). Hence, they can be expected to remain stable in the comparatively mild conditions of 3 M NaSCN. The high level of succinimide on the support is intended to insure that a large fraction of F1 molecules are covalently linked to the support, and not retained merely through interaction with other bound F1 molecules. Prior to use of the affinity columns for antibody purification, the columns are treated with 3 M NaSCN to elute unstable F1. If large amounts of F1 are released by this treatment reducing column capacity to unacceptable levels, alternative methods are used to stabilize F1 in the column. One approach is cross-linking F1 to the support in a monomeric state. F1 is treated at high temperature in SDS to disrupt the oligomers, and the cross-linked in the presence of SDS to prevent reoligomerization. The succinimide cross-linker is highly specific for primary amines and sulfhydryl groups, and is relatively unaffected by the detergent. Following removal of SDS, F1 denatured by this method is known to refold and oligomerize, so renaturation of the support-bound F1 is expected. Alternatively, oligomeric F1 is stabilized by treatment with glutaraldehyde prior to reaction with the support. Since both of these alternatives disrupt the conformation of F1 to some degree, direct attachment of native oligomeric F1 is preferred.

After a stable affinity column is prepared, antibody is purified by established methods, using 3 M NaSCN for elution as noted above. Procedures include washing of the column with 1 mM NaSCN after binding of the antibody to elute non-specifically bound proteins, and rapid removal of NaSCN from eluted antibody on a desalting column to limit denaturation. If the use of NaSCN elution proves unsuitable for any reason, alternative methods (high Mg⁺⁺, low pH) can be employed.

As noted above, antibodies can be similarly prepared that are immunoreactive to other biological agent antigens. Suitable alternative antigens include V antigen, YpkA, YopH, YopM, YopB, YopD, YopN, YopE, YopK, Pla, pH 6 antigen and purified LPS (Benner, G. E., et al., Immune response to Yersinia outer proteins and other Yersinia pestis antigens after experimental plague infection in mice, Infect Immun. 1999 April; 67(4):1922-8). Preferred alternative antigens include V antigen, YopH, YopM, YopD and Pla. A preferred alternative Y. pestis antigen is the Pla antigen.

Control Antibody that is Specific for an Irrelevant Protein.

Samples taken by throat swab contain many other bacteria, human cells and cell debris, proteins contained in pharyngeal mucus, and other biological materials. Specific detection of Y. pestis in this environment through the use of specific antibody is facilitated by use of an internal control antibody preparation that does not recognize components of the sample and can thus provide a measure of non-specific antibody binding. It is important that this nonspecific antibody preparation be as similar as possible to the specific antibody preparation, so that nonspecific binding by both preparations is similar. In one embodiment, monoclonal antibodies are used to detect the pathogen, and a mixture of monoclonal antibodies of the same class and subclass as the specific monoclonal antibodies is used at the control.

Alternatively, the control antibody is a preparation of polyclonal rabbit antibody prepared by methods very similar to those employed for the F1 specific polyclonal preparation is needed if a F1 specific polyclonal is used. A commercially available antibody preparation that meets these latter requirements is an antibody that is specific for green fluorescent protein (GFP) of the jellyfish Aequorea Victoria, (NB 600-310, Novus Biologicals, Littleton, Colo.). This antibody is unlikely to react specifically with proteins in throat swab samples. It is a polyclonal rabbit antibody purified first on Protein A, and then by affinity chromatography against GFP. This purification parallels the above scheme for anti-F1, and thus yields a similar mix of antibody types adequate for non-specific binding control.

Western blotting is used to confirm that minimal specific binding and similar degrees of nonspecific binding to components of throat swab sample occurs with both the anti-GFP control antibody and the anti-F1 antibody. Material from throat swabs obtained from five individuals is separated by SDS-PAGE and identical blots containing all five sample preparations are stained with each of the antibody preparations.

Phage Display Subtraction is Used to Isolate Antibodies to Non-F1 Unique Epitopes on the Pathogen Surface

One approach is selection of antibody fragments from a nonimmune phage display antibody repertoire against one set of antigens in the presence of a competing set of antigens (Stausbol-Grøn, B., et al., De novo identification of cell-type specific antibody-antigen pairs by phage display subtraction. Isolation of a human single chain antibody fragment against human keratin 14. Eur J Biochem 2001 May; 268(10):3099-107). This is performed in order to enrich phage antibodies binding to keratinocyte-specific antigens while avoiding those binding to common antigens between keratinocytes and melanoma cells. This approach is used to produce phage antibodies directed against non-F1 Y. pestis-specific antigens without knowing the identity of these latter antigens beforehand. Furthermore, the isolated phage antibodies themselves could serve as probes for the identification of their cognate antigens by 2D PAGE immunoblotting and search in a 2D PAGE database.

Phage Display Antibody Repertoire and Bacterial Strains

The protocol in general is based on that described by Stausbol-Grøn, B., et al., 2001. Briefly, an nonimmunized semisynthetic phage display antibody repertoire (Griffin. 1) is used. The repertoire is a single chain Fv (scFv) phagemid repertoire constructed by recloning the heavy and light chain regions from the lox library (Griffiths, A. D., et al. (1994) Isolation of high affinity human antibodies directly from large synthetic repertoires. EMBO J. 13, 3245-3260.). Escherichia coli TG1 (supE hsdD5 Δ(lac-proAB) thi F′ {traD36 proAB+lacI^{q} lacZΔM15]) is an amber suppressor strain (supE) and is used for propagation of phage particles. E. coli HB2151 (ara Δ(lac-proAB) thi F′ {proAB+lacI^{q} lacZΔM15]) is a nonsuppressor strain and is used for expression of soluble scFv.

Direct Selection of scFv Against Antigens Such as F1

A suitable protocol is described by Stausbol-Grøn, B., et al., 2001. Briefly, an immunotube (Nunc, Maxisorp®) is coated overnight with purified antigens in 4 mL 50 mM NaHCO3 pH 9.6 at 4° C. overnight. The coated immunotube is washed three times with NaCl/Pᵢ, and then is blocked for 1-2 h at 30° C. in 2% skimmed milk powder in NaCl/Pᵢ (NaCl/Pᵢ/milk), added to the brim. For the 1st round of selection, about 10¹³ phage particles from the repertoire stock are preblocked for 30 min with 4 mL 2% NaCl/Pᵢ/milk in a Falcon 2059 tube. The blocked immunotube is washed three times with NaCl/Pᵢ. Subsequently, the preblocked phage particles are added to the immunotube and incubated for about 90 minutes at room temperature. The immunotube is then washed 20 times with NaCl/Pᵢ/0.2% Tween-20 (NaCl/Pᵢ/Tween) and 20 times with NaCl/Pᵢ. Thereafter, the bound phage particles are eluted and amplified as described by Marks, J. D., Hoogenboom, H. R., Bonnert, T. P., McCafferty, J., Griffiths, A. D. & Winter, G. (1991) By-passing immunization. Human antibodies from V-gene libraries displayed on phage. J. Mol. Biol. 222, 581-597. For the subsequent rounds of selection, 3 mL cleared culture supernatant of the overnight culture containing phage particles from the previous round of selection plus 1 mL 8% NaCl/Pᵢ/milk is used as the input.

Subtractive Strategy for Selection of scFv Against Non-F1 Antigens

Coating is performed with 25 μg·mL⁻¹ of antigens in 50 mM NaHCO₃, pH 9.6 overnight at 4° C. and blocking with 4% NaCl/Pᵢ/milk at 30° C. for about 1 hour. For the first round of selection, about 10¹³ phage particles from the library stock, and soluble competitive F1 antigens in 4 mL 2% NaCl/Pᵢ/milk at a concentration of 25 μg·mL⁻¹, are added to an immunotube (about 1885 mm²) (Nunc, Maxisorp®) coated with competitive F1 antigens. The phage particles are subjected to a 30-min preincubation at room temperature. In parallel, immunobeads coated with non-F1 antigens such as the Pla antigen are preincubated with polyclonal anti-F1 scFv (>100 μg·mL⁻¹) in order to mask common epitopes. Subsequently, the masked immunobeads are washed briefly in NaCl/Pᵢ and added to the immunotube followed by incubation for 90 min at room temperature. The immunobeads are washed 20 times in NaCl/Pᵢ/Tween and 20 times in NaCl/Pᵢ. Next, the immunobeads are pre-eluted with polyclonal anti-F1 scFv (>100 μg·mL⁻¹) for 30 min. After a brief wash in NaCl/Pᵢ, the phage are eluted and amplified as described in Marks et al., 1991. For the subsequent rounds of selection, about 3 mL cleared culture supernatant of the overnight culture containing phage particles from the previous round of selection plus 1 mL 8% NaCl/Pi/milk is used as the input.

Preparation of Phage Particles from Single Clones

Individual colonies from TYE agar plates containing 100 μg·mL⁻¹ ampicillin and 2% glucose are picked with a sterile toothpick into 100 μL 2TY containing 100 μg·mL⁻¹ ampicillin and 2% glucose in a 96-well polypropylene microtiter plate. Phage particles are produced by superinfection with the helper phage M13KO7 (Pharmacia) as described in Marks et al., 1991. The cleared supernatants of the culture containing the phage particles produced by the single clones are used directly as reagents.

Expression and Preparation of scFv

Soluble scFv fragments are obtained from the periplasmic fractions from 20 mL cultures of infected E. coli nonsuppressor strain HB2151 induced with 1 mM isopropyl thio-beta-D-galactoside, and grown for 4 h at room temperature with shaking. Pellets of the bacterial cultures are resuspended in 300 μL Mops buffer (20 mM Mops pH 7.5, 0.5 mM EDTA, 20% sucrose) and left on ice for 15 min. Subsequently, 1.6 mL water is added to disrupt the outer bacterial membrane. The suspensions are centrifuged at 3000 g for 20 min at 4° C., and the supernatants are used as reagents.

Bio-Panning Against Biological Agents.

In another embodiment, a human single-chain Fv (scFv) library can be amplified and rescued, as described (Gao, at al., Making chemistry selectable by linking it to infectivity, Proc. Natl. Acad. Sci. USA, Vol. 94, pp. 11777-11782, October 1997). The library is panned against biological agents suspended in PBS (10 mM phosphate, 150 mM NaCl, pH 7.4) and the positive scFv-phage are selected against immobilized spores by enzyme-linked immunosorbent assay (ELISA). Briefly, the purified scFv-phage are incubated with abort 5×10⁷ biological agents in PBS containing 1% (w/v) bovine serum albumin (BSA) for 2 hr at room temperature with occasional shaking. After this time, the biological agents are spun down at full speed in a microcentrifuge for 5 min and then are resuspended thoroughly in 1.5 ml of PBS containing 1% (w/v) BSA and washed by pipetting up and down. This centrifugation-resuspension-washing cycle is repeated several times. Then, the pellet is resuspended in 200 μl of acid elution buffer (100 mM glycine-HCl, pH 2.2, 1% BSA), incubated at room temperature for 10 min, followed by neutralization with 12 μl of 2 M Tris base. The biological agents are spun down and the supernatant containing the eluted phage is transferred into a fresh tube and mixed with 2 ml of freshly prepared XL1-Blue cells (Stratagene) and incubated at 37° C. for 1 hr. An aliquot is removed for titration of the eluted phage and the remainder is plated onto a gctSB (10 g of 3-N-morpholino-propanesulfonic acid, 30 g of tryptone, and 20 g of yeast extract per liter, with 2% (w/v) glucose, 50 μg/ml carbenicillin and 10 μg/ml tetracycline) agar plate and incubated overnight at 30° C. The plate is flooded with pre-warmed gctSB medium, the cells carefully suspended with a glass spreader, and the suspension harvested and resuspended thoroughly by vortexing. A 0.50-ml aliquot of suspension is used to inoculate 100 ml of gctSB; the aliquot is shaken at 300 rpm at 37° C. until the OD₆₀₀≈0.5. VCSM13 helper phage (Stratagene) are added to a final concentration of 10¹⁰ pfu/ml and shaken at 200 rpm for 2 hr at 37° C. The infected culture is transferred into a 500-ml centrifuge tube and centrifuged at 8,000×g for 10 min at 4° C. The cell pellet is resuspended into 100 ml of SB medium supplemented with 100 μg/ml carbenicillin, 70 μg/ml kanamycin, 10 μg/ml tetracycline, and 1 mM IPTG, and shaken at 300 rpm overnight at 30° C. Then, the cells are centrifuged at 8,000×g for 10 min at 4° C., the bacterial pellet is saved for phagemid DNA preparation, the supernatant is transferred to a clean 500 ml bottle, and 4% (w/v) PEG-8000 and 3% (w/v) of NaCl are added. The solids are dissolved by shaking and the mixture is incubated on ice for at least 1 hr. After centrifugation at 10,000×g for 15 min at 4° C., the supernatant is discarded and the bottle is drained by inverting on a paper towel for at least 10 min, wiping the remaining liquid from the neck of the bottle with a paper towel. The phage pellet is resuspended in 2 ml of PBS containing 1% (w/v) BSA by pipetting up and down along the side of the centrifuge bottle. The suspension is transferred to a 2-ml microcentrifuge tube and spun at full speed in a microcentrifuge for 10 min, and then the supernatant is transferred to a fresh tube. The above round of panning is repeated for another 3-5 rounds until the phage output titer reached 10⁹-10¹⁰ cfu/ml. After the last round of panning, scFv-phage are selected from the pool by randomly picking 48 clones that are inoculated individually into wells of a microtiter plate. The scFv-phage are rescued as described above, and positive clones are selected by ELISA.

A solution of 1×10⁷ colony-forming units (cfu)/ml prepared from the stock solution is washed twice with PBS and centrifuged; the pellet is resuspended in PBS at a concentration of 1×10⁷ cfu/ml. An aliquot of suspension (25 μl per well) is added to Maxisorp (Corning) microtiter plates and incubated overnight at 4° C. The coated plates are washed twice with PBS and blocked with Blotto solution (5% skimmed milk in PBS) for 1 h at room temperature. Then, 25 μl per well of scFv-phage are added at different concentrations (2-fold serial dilution) and incubated 1 h at room temperature. After the plate is washed 8-10 times with PBS, 25 μl per well of horseradish peroxidase-conjugated mouse anti-M13 antibody (Amersham Pharmacia) diluted 1:1,000 in Blotto was added and incubated for 1 h at room temperature. The plate is washed 10 times with PBS, then 50 μl per well of TMB/H₂O₂ substrate solution (Pierce) is added and incubated at room temperature until an adequate signal was reached. The reaction is stopped by the addition of 50 μl per well of 2 M.H₂SO₄, and the absorbance is measured at 450 nm with a Thermomax microplate reader (Molecular Devices).

Chain Shuffling and Subtractive Panning.

The heavy chain variable region (V_{H}) and light chain variable region (V_{L}) fragments from nine positive scFv clones are individually amplified by PCR; V_{H} and V_{L} then are separately pooled. A Fab sublibrary is constructed from these pooled V_{H} and V_{L} fragments, and the Fab-phage are rescued, as described above. For construction of the Fab chain-shuffled library, the V_{H} and V_{L} fragments from selected scFv clones are assembled into Fab inserts in the orientation Of V_{H}-C_{H1}-ompA-leader-V_{L}-Ck by PCR. The Fab fragments are digested by the restriction enzyme Sfi I and then are cloned into a Sfi I digested pCGMT vector [Gao, C., Lin, C.-H., Lo, C.-H. L., Mao, S., Wirsching, P., Lerner, R. A. & Janda, K. D. (1997) Proc. Natl. Acad. Sci. USA 94, 11777-11782].

Two rounds of regular panning are carried out to enrich all of the possible binders. For the third round of panning, purified Fab-phage are first incubated with, e.g., about 1×10⁹ Y. pestis expressing F1 for 2 h at room temperature to subtract binders from the pool that recognize a common epitope on both Y. pestis expressing F1 and Y. pestis not expressing F1. The biological agents are spun down, and the supernatant is transferred to about 1×10⁷ Y. pestis expressing F1 and incubated for another 2 h at room temperature. The washing, elution, and amplification procedure is as described above for bio-panning. Additional rounds of subtractive panning are performed until the output titer reaches 10⁹-10¹⁰ cfu/ml. The positive clones are selected by ELISA.

Characterization of Antibody-Phage by Competition ELISA.

The specificity of individual selected scFv-phage clones and Fab-phage clones for free pathogens in solution can be assessed by various types of competition ELISA. The concentration or dilution factor of each antibody-phage is determined by titration on a microtiter plate coated with a given pathogen. Competition ELISA using plates coated with various pathogen strains is performed as follows. The purified strains are prepared in PBS at the concentration of 1×10⁷ cfu/ml. Each column on the plate is coated with a different strain and then is washed and blocked as described for ELISA. Then, antibody-phage from the stock is prepared at twice the assay concentration (determined above by serial dilution) in PBS. In a 0.50-ml tube, equal volumes of a given strain suspension (1×10⁷ cfu/ml) and antibody-phage solution are mixed and incubated at room temperature for 1 h with occasional shaking. The pathogens are spun down at full speed in a microcentrifuge for 5 min, then 25 μl per well of the supernatant is added to the pathogen-coated ELISA plates (25 μl per well of VCSM13 helper phage can be used as a control) and incubated at room temperature for 1 h. The remainder of the ELISA procedure is as described. The relative binding capacity and specificity of the different antibody-phage for a specific pathogen can be determined by using 3336 coated plates as follows. The plate is coated with 1×10⁷ cfu/ml pathogen in PBS, washed, and blocked as described above. A diluted antibody-phage solution is prepared at the assay concentration (determined by serial dilution) in PBS. A 0.50-ml tube containing 1×10⁷ cfu/ml pathogens is washed with PBS, spun down, and the pellet resuspended in 400 μl of a high concentration of antibody-phage stock solution (not diluted) and incubated at room temperature for 1 h with occasional shaking. The pathogens are washed several times with PBS and spun down; the pathogen pellet is resuspended in 100 μl of diluted antibody-phage solution and incubated at room temperature for 1 h with occasional shaking. The pathogens are spun down, then 25 μl per well of supernatant is added to a pathogen-coated plate and is incubated at room temperature for 1 h. The remainder of the ELISA procedure is as described above.

Fluorophores

In preferred embodiments, specific antibodies are conjugated to fluorophores that can be distinguished by their excitation spectra, their emission spectra or both. In preferred embodiments, fluorophores for multi-wavelength emission have distinct emission spectra with minimal overlap (i.e. large Stokes shifts), emit at wavelengths at which silicon detectors have good efficiency, have reasonable quantum efficiency; and are excitable by low cost light sources.

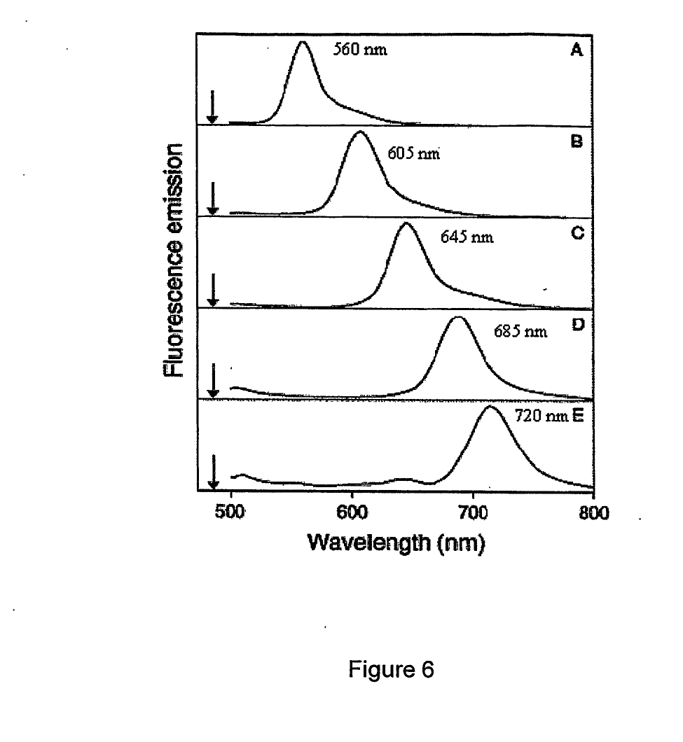

Suitable fluorophores in particulate form include TransFluoSpheres™, available from Molecular Probes (Eugene, Oreg.). The emission spectra of five examples are shown diagrammatically in FIG. 6. The TransFluoSpheres™ particles are microspheres that contain two or more fluorophores per particle that are suited for detecting multiple biological agents in a single assay. As illustrated in FIG. 3, the five fluorescent particles have distinctly different emission maximum wavelengths (λₘₐₓ) at 560 nm, 605 nm, 645 nm, 685 nm, and 720 nm, respectively, and have a common excitation maximum at 488 nm, so all can be excited with a light source such as a xenon arc lamp, xenon flash lamp, light emitting diode (LED), laser diode or laser. Such fluorescent particles can be conjugated to antibodies and used in a two-particle sandwich assay where the only signal that is detected is from antigen that is bound both with the TransFluoSpheres™ label, and the paramagnetic capture particle (i.e. carried into the evanescent wave).

Other fluorophores suitable for labeling proteins such as antibodies and available commercially are identified in Table 4, below. The listed absorption maxima and emission maxima are those of the protein conjugates.

TABLE 4
Approximate spectral properties of the protein conjugates of
amine-reactive fluorophores
(Molecular Probes, Eugene OR, http://www.probes.com/handbook/
tables/0726.html)

| | Abs | Em |
|---|---|---|
| Fluorophore | (nm) | (nm) |
| Alexa Fluor 350 | 346 | 442 |
| Alexa Fluor 405 | 402 | 421 |
| Alexa Fluor 430 | 433 | 539 |
| Alexa Fluor 488 | 495 | 519 |
| Alexa Fluor 500 | 503 | 525 |
| Alexa Fluor 514 | 518 | 540 |
| Alexa Fluor 532 | 531 | 554 |
| Alexa Fluor 546 | 556 | 575 |
| Alexa Fluor 555 | 555 | 565 |
| Alexa Fluor 568 | 578 | 603 |
| Alexa Fluor 594 | 590 | 617 |
| Alexa Fluor 610 | 612 | 628 |
| Alexa Fluor 633 | 632 | 647 |
| Alexa Fluor 647 | 650 | 668 |
| Alexa Fluor 660 | 663 | 690 |
| Alexa Fluor 680 | 682 | 702 |
| Alexa Fluor 700 | 696 | 719 |
| Alexa Fluor 750 | 752 | 779 |
| AMCA | 349 | 448 |
| BODIPY 493/503 | 500 | 506 |
| BODIPY FL | 505 | 513 |
| BODIPY R6G | 528 | 550 |
| BODIPY 530/550 | 534 | 554 |
| BODIPY TMR | 542 | 574 |
| BODIPY 558/568 | 558 | 569 |
| BODIPY 564/570 | 565 | 571 |
| BODIPY 576/589 | 576 | 590 |
| BODIPY 581/591 | 584 | 592 |
| BODIPY TR | 589 | 617 |
| BODIPY 630/650 | 625 | 640 |
| BODIPY 650/665 | 646 | 660 |
| 6-Carboxy-2′,4,4′,5′,7,7′- | 535 | 556 |
| hexachlorofluorescein, | | |
| succinimidyl ester (6-EX, SE) | | |
| 6-Carboxy-2′,4,7,7′- | 521 | 536 |
| tetrachlorofluorescein, | | |
| succinimidyl ester (6-TET, SE) | | |
| Cascade Blue dye | 400 | 420 |
| Cascade Yellow dye | 402 | 545 |
| Dansyl | 340 | 520 |
| Dapoxyl dye | 373 | 551 |
| 4′,5′-Dichloro-2′,7′- | 522 | 550 |
| dimethoxy-fluorescein | | |
| 2′,7′-Dichloro-fluorescein | 510 | 532 |
| Eosin | 524 | 544 |
| Erythrosin | 530 | 555 |
| Fluorescein | 494 | 518 |
| Hydroxycoumarin | 385 | 445 |
| Lissamine rhodamine B | 570 | 590 |
| Marina Blue dye | 365 | 460 |
| Methoxycoumarin | 340 | 405 |
| Naphthofluorescein | 605 | 675 |
| NBD | 465 | 535 |
| Oregon Green 488 | 496 | 524 |
| Oregon Green 514 | 511 | 530 |
| Pacific Blue dye | 410 | 455 |
| PyMPO | 415 | 570 |
| Pyrene | 345 | 378 |
| Rhodamine 6G | 525 | 555 |
| Rhodamine Green dye | 502 | 527 |
| Rhodamine Red dye | 570 | 590 |
| 2′,4′,5′,7′- | 528 | 544 |
| Tetrasbromosulfonefluorescein | | |
| Tetramethyl-rhodamine (TMR) | 555 | 580 |
| Texas Red dye | 595 | 615 |
| X-rhodamine | 580 | 605 |

Alternatively, the fluorophores can be derivatized quantum dots. Suitable derivatized quantum dots fluorophores are commercially available from Quantum Dot Corp, Hayward, Calif., USA or Evident Technologies, Inc., Troy, N.Y., USA. Quantum dots are semiconductor nanocrystals about 2-30 nanometers in size, that typically have broad excitation spectra and narrow emission spectra. The emission spectrum can be tuned independently of the excitation spectrum by changing the size of the quantum dots, allowing the use of multiple fluorophores that have overlapping excitation spectra but spectrally distinguishable emission spectra. See Jaiswal, J. K., et al., Long-term multiple color imaging of live cells using quantum dot bioconjugates, Nat Biotechnol. 2003, 21 (1):47-51; Goldman, E. R., et al., Avidin: a natural bridge for quantum dot-antibody conjugates, J Am Chem Soc. 2002, 124(22):6378-82. For example, CdSe provides emission on the visible range, CdTe in the red near infrared, and InAs in the near infrared (NIR). The size is then used to fine-tune the exact wavelength desired so that 3 nm CdSe produces 520 nm emission, 5.5 nm CdSe produces 630 nm emission, and intermediate sizes result in intermediate colors. The emission width is controlled by the size distribution, so a very monodisperse samples can have emission-peak full widths at half max (FWHM) in the 20-35 nm range. Quantum dot fluorophores typically have broad excitation spectra that peak at wavelengths shorter than 400 nm. Excitation is typically accomplished in the range of about 340 nm to about 460 nm. In some embodiments, the xcitation light can be a 40-50 nm bandwidth centered at 425-450 nm. or a laser at about 450-514 nm. Quantum dot fluorophores typically have higher extinction coefficients than organic dye fluorophores. For example, Qdot 605 Streptavidin Conjugates (Quantum Dot Corp., Hayward, Calif., USA) have an extinction coefficient of approximately 650,000 M⁻¹cm⁻¹ at 600 nm, increasing to around 3,500,000 M⁻¹cm⁻¹ at 400 nm, and even higher at shorter wavelengths (compared to fluorescein, which is around 80,000 M⁻¹cm⁻¹ at its peak). Such quantum dot conjugates are efficiently excited by 568-nm, 532-nm, 488-nm, 457-nm, 405-nm, and UV lasers. Preferred light sources include 405-nm, 457 nm and 488 nm lasers. Watson, A., et al., Lighting up cells with quantum dots, BioTechniques 2003 34: 296-303.

In general, in the system of the present invention, a fluorophore, such as that having an emission spectrum shown in FIG. 6A, is routinely used to label the control antibody that is specific for an irrelevant protein. In the terminology used above, this “second fluorophore” used to label the control antibody would have a consistent peak emission wavelength.

In such a system, a series of fluorophores suitably would have a peak emission wavelength in the range of about 550 nm to about 580 nm; about 590 nm to about 630 nm, preferably about 585 nm to about 615 nm; about 630 nm to about 660 nm, preferably about 635 nm to about 655 nm; about 660 nm to about 715 nm, preferably about 675 nm to about 695 nm; and about 690 nm to about 740 nm, preferably about 710 nm to about 730 nm.

Fluorophore labeling of antibodies is carried out as follows. TransFluoSpheres™ contain pendant carboxylic acids that can be conjugated to amine functional groups on the antibody using carbodiimide reagents such as 1-ethyl-3-(3-dimethylaminopropyl carbodiimide hydrochloride (EDAC) which results in an amide linkage. For example, the 488/560 TransFluoSphere™ (T-8864) is conjugated to anti-F1-IgG using EDAC as a crosslinker thereby generating the anti-F1/T-8864 antibody-fluorophore conjugate. Similarly, the 488/720 TransFluoSphere™ (T-8869) is conjugated to anti-GFP-IgG (internal control conjugate) using EDAC as a crosslinker thereby generating the anti-GFP/T-8869 antibody-fluorophore conjugate. The efficiency of labeling of Yersinia pestis cells is optimized by testing anti-F1/T-8864 conjugates made using different proportions of fluorophore and antibody. Fluorophore/antibody ratios are determined by spectral analysis and protein assay. The efficiency of labeling of Yersinia pestis by the anti-F1 conjugates is determined by measuring the fluorescence of intact bacteria bound to the wells of micro titer plates following treatment with fixed amounts of anti-F1 conjugates with different antibody/fluorophore ratios using a fluorescence microplate reader with tunable excitation and detection frequencies. Similarly, an optimized conjugate/antibody ratio for anti-GFP/T-8869, i.e., a ratio that provides good fluoresence but is unlikely to interfere with antibody specificity, is determined using the optimized antibody/fluorophore ratio determined for the anti-F1/T-8864 conjugate. The underlying assumption is that the degree of conjugation optimal for anti-F1/T-8864 is unlikely to have a deleterious effect on the properties of anti-GFP.

In another embodiment, the fluorophores are not particles. Suitable fluorescent labels are available from Molecular Probes (Eugene, Oreg.) alone or conjugated to various antibodies: 1) Alexa Fluor 488 goat anti-mouse IgG antibody (λₘₐₓ about 519 nm), 2) R-phycoerythrin goat anti-mouse IgG antibody (λₘₐₓ about 565 nm), 3) Alexa Fluor 610-R-phycoerythrin goat anti-mouse IgG antibody (λₘₐₓ about 627 nm), 4) Alexa Fluor 647-R-phycoerythrin goat anti-mouse IgG antibody (λₘₐₓ about 667 nm) and 5) Alexa Fluor 680-R-phycoerythrin goat anti-mouse IgG antibody (λₘₐₓ about 702 nm). The phycobiliprotein R-phycoerythrin (R-PE) has efficient excitation at 488 nm and emission at 578 nm. By conjugating R-PE to longer wavelength light-emitting fluorescence acceptors, an energy transfer cascade is established wherein excitation of the R-PE produces fluorescence of the acceptor dye by the process of fluorescence resonance energy transfer (FRET). This process can be quite efficient, resulting in almost total transfer of energy from the phycobiliprotein to the acceptor dye of these “tandem conjugates.” Typically, in such a system, a series of antibodies labeled with fluorophores suitably would have a peak emission wavelength in the range of about 500 nm to about 540 nm; about 560 nm to about 590 nm; about 610 nm to about 640 nm; about 650 nm to about 680 nm; and about 690 nm to about 720 nm.

In one embodiment, a first fluorescent label with a first maximum emission wavelength is used to label an antibody specific for first biological agent (e.g., for a plague assay, conjugated to an anti-F1-IgG), and a second fluorescent label with a second maximum emission wavelength is used to label an antibody that acts as an internal control to correct for any non-specific binding (i.e. conjugated to anti-GFP-IgG). In other embodiments, the system further comprises a third fluorescent label with a third maximum emission wavelength used to label an antibody specific for a second biological agent, preferably further comprises a fourth fluorescent label with a fourth maximum emission wavelength used to label an antibody specific for a third biological agent, and more preferably further comprises a fifth fluorescent label with a fifth maximum emission wavelength used to label an antibody specific for a fourth biological agent. In a preferred embodiment, the biological agents are Category A agents.

In a preferred embodiment the fluorophores are excited by light from an excitation light source selected from the group consisting of xenon arc lamp, xenon flash lamp, light emitting diode (LED), laser diode or laser. Suitable excitation light sources having light output at 440-488 m are listed in Table 1, above. Other light source and fluorophore combinations are known, for example, in the art of flow cytometry.

Selection and preparation of paramagnetic beads for the capture of Yersinia pestis. Paramagnetic beads with the ability to specifically capture Yersinia pestis from dispersed throat swab samples play two critical roles in detection. First, they effectively sweep through the entire sample volume, capturing a significant fraction of the Yersinia pestis cells (and soluble F1 oligomers) present. Secondly, they concentrate the captured bacteria into the thin zone on the sample cell surface where excitation energy is available to the fluorophores, and where they are in the focal plane of the fluorescence spectrometer that serves as the detector. This capture-and-deliver-to-the-excitation-zone role not only contributes to the specificity of the assay, but also greatly simplifies the detection problem by limiting background fluorescence: fluorophore on unbound antibody in the bulk of the sample receives minimal excitation and hence produces virtually no fluorescence.

The paramagnetic beads used must be small enough to deliver a significant fraction of their load into the optical excitation zone, which is on the order of 0.5 μm thick. They must be large enough and have a content of paramagnetic material sufficient to permit rapid collection in the excitation zone after the incubation period for capture is complete. They must also have a surface chemistry that will permit covalent coupling of antibody, and the surface must be treated after coupling of the antibody to prevent non-specific binding.

Suitable beads having an approximate diameter of 1 μm, a magnetite content of 60%, and a —NH₂ modified surface are obtained commercially from Bangs Laboratories, Fishers, Ind. (Cat #MCO5N/2315). The size of these beads is similar to that of the bacteria, and is small enough to remain in suspension without mixing during the capture period. However, with their high magnetite content it is estimated that the majority of the beads can be drawn into the excitation zone in 2-3 minutes in a magnetic field achievable with rare-earth magnets of modest size and cost.

Anti-F1 is covalently cross-linked to the bead surface using the water-soluble homo-bifunctional amine-reactive cross-linking reagent bis[sulfosuccinimidyl]subarate (Pierce). Efficiency of cross-linking is monitored by measuring unbound protein. Following antibody coupling, the beads are incubated in BSA to block non-specific binding to the bead surface. The ability of the beads to agglutinate Yersinia pestis is used as a preliminary test to insure that the coupled antibody remains functional.

Assessment of anti-F1/AF633 as a specific labeling reagent is performed as follows. Yersinia pestis KIM5 is incubated in anti-F1/T-8864 at selected concentrations, collected and washed by rapid centrifugation, and the intensity of fluorescence labeling of individual bacteria determined by analysis of images collected with a cooled integrating CCD camera and fluorescence microscope. The incubation period for labeling can be fixed at 15-20 minutes at room temperature. Typically it can be anticipated that concentrations of about 10 μg/ml and lower are useful for labeling.

Once a practical antibody concentration has been selected, reconstruction experiments in which Yersinia pestis KIM5 added to throat swab samples are used to confirm adequate specific labeling under more realistic conditions. The bacteria are applied in a small volume onto freshly collected throat swabs and incubated for 10 minutes to allow absorption of material from the sample on to the Yersinia pestis surface and possible absorption of Yersinia pestis to the swab. The swab is then agitated in a 0.5 ml volume of buffer to disperse the sample. The resulting suspension is labeled with antibody and examined as described above. To test for non-specific labeling, sample swabs without added Yersinia pestis are processed in the same way.

An important aspect of the system is the method of coupling excitation light onto the surface of the inside of the sample chamber. Means of coupling light to the wall of a glass or optical polymer container are known in the art. The coupling can be accomplished either by the use of refractive or reflective optical elements (lenses and mirrors) as shown in FIG. 2 or optical fibers as shown in FIG. 3 (for example, a fiber bundle, with the fibers bundled at the emitter end and spread over a rectangular area at the glass tube end). The excitation-light-receiving end of a glass container can be ground and polished to enable maximum light coupling. Once coupled into the glass container wall, the evanescent optical wave propagating along the outside of the cell wall produces the fluorescent emission from the fluorophores. Diodes and diode lasers having emission spectra that match the spectral absorption of the fluorophores, having an output of several mW are suitable (see Table 2, above). Optical filters 430 with spectral transmission characteristics matched to the emission from the fluorophores and dichroic mirrors 420 can be used to minimize the amount of excitation light reaching the photodetector.

Optimization of antibody-coupled paramagnetic beads as a capture reagent is initially tested by incubation with bacteria at a selected series of bacterial densities, followed by magnetic harvesting of the beads and determination of number of bacteria remaining in suspension by dilution and plating. The results are used to determine the density of beads required to yield a given capture efficiency in a fixed incubation period of 15-20 minutes.

Typically a magnetic field of several thousand Gauss is adequate for directing the paramagnetic microbeads to the fluorescence-detecting side of the sample chamber. Such a magnetic field intensity can be obtained from off-the-shelf permanent magnets available from a number of commercial sources. A gaussmeter can be used for generating magnetic field uniformity plots and the magnets adjusted to create the maximum magnetic force on the paramagnetic particles. Performance can be optimized by measuring the length of time required for all magnetic particles to accumulate at the desired sample chamber wall area.

For example, recognition of a sample as positive for Yersinia pestis can be accomplished in two distinct modes. If few Yersinia pestis cells are present, the image of the surface layer in which in which the paramagnetic beads are concentrated and where excitation of the fluorophores occurs contains a number of high-intensity pixels when viewed through a filter specific for the emission from the fluorophore used to label anti-F1, but which are of comparatively low intensity when viewed through a filter specific for emission from the fluorophore used to label anti-GFP. These high intensity pixels are illuminated by emission from individual Y. pestis cells. Counting of high intensity pixels in some form is required under these circumstances. When many Yersinia pestis cells are present, emission by the anti-F1 fluorophore may be sufficiently strong that a simple comparison of the average intensity seen through both filters is sufficient to provide robust determination that the sample is positive. In laboratory cultures, roughly 50% of F1 is in a soluble form not associated with the bacteria. If this is also true during growth in the pharynx, significant fluorescence may be contributed by antibody bound soluble F1 oligomers that have been captured by the paramagnetic beads. When few Yersinia pestis cells are present, the concentration of soluble F1 will be low, and individual beads will not be intensely fluorescent unless they have captured a bacterium.

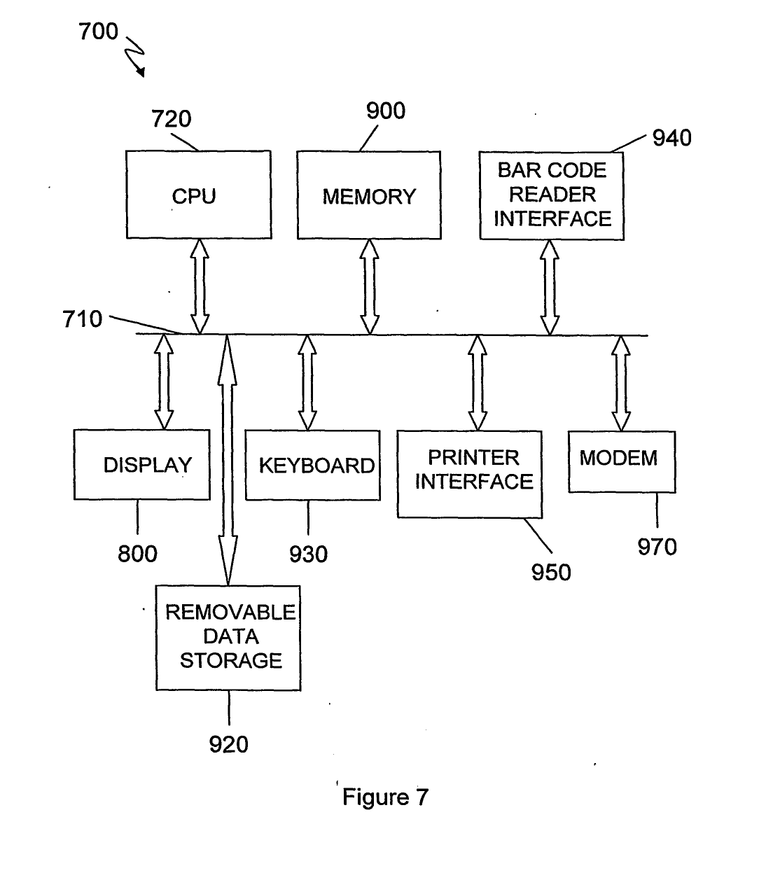

FIG. 7 is a schematic diagram of one embodiment of an image analyzer 700. A bus 710 connects a central processing unit (CPU) 720, display 800, memory 900, removable data storage 920, keyboard 930, bar code reader interface 940, printer interface 950, and modem 970. The internal memory 900 is preferably a non-volatile form of memory. In one embodiment adapted to testing in the field, the keyboard 930 is a sealed membrane keyboard. In one embodiment, the keyboard 930 is a full ASCII keyboard. In other embodiments, the keyboard includes special purpose function keys.

In some embodiments, the display 800 is a liquid crystal display (LCD), preferably capable of displaying text and graphical icons. In some embodiments, the display 800 supports a graphical user interface (GUI) and can display the images produced by the CCD. A preferred operating system is a Windows® operating system such as Windows® 98, Windows® NT, Windows® 2000, Windows® XP or Windows® CE.

The bar code reader interface 940 permits the entry of bar coded information that can be used to identify the pre-filled sample chamber, patient and other information. In some embodiments, sample chambers are identified with regard to reagents contained by a bar code that is automatically ready by an internal bar code reader when the sample chamber is inserted. In other embodiments, an external bar code reader is available to read identifying codes from a patient's wrist band and chart. This information is collected in a record associated with the test results.

The keyboard 930 is also used to enter data. The bar code reader interface 940 is connected to a bar code reader by a cable, or optionally, by a local wireless means, such as those supporting the Bluetooth protocol.

Printer interface 950 is connected to a printer by a cable, infrared link or optionally, by a local wireless means, such as those supporting the Bluetooth protocol. The printer preferably supports printing graphics, including bar codes. In one embodiment, the printer is integrated into the unit housing the biological agent detector and the image analyzer. In another embodiment, the printer is in a separate unit.

Removable data storage 920 provides a means for storing and transferring test results. In some embodiments the removable data storage 920 provides a means for storage and transfer of programs. In preferred embodiments, the removable data storage 920 is a solid state device, such as compact flash card, secure digital card or Memory Stick®.

In one embodiment, modem 970 is a wireless modem or a modem connected to a local area network or the telephone system. The modem 970 provides access to a remote computer via the Internet or a local area network (LAN). FIG. 5 is a schematic diagram of an embodiment of a system comprising a biological agent detector 100 operatively connected to an image analyzer 700 that is in turn connected to a remote computer 640 by connections through a local area network (LAN) 620 and through the Internet 600.

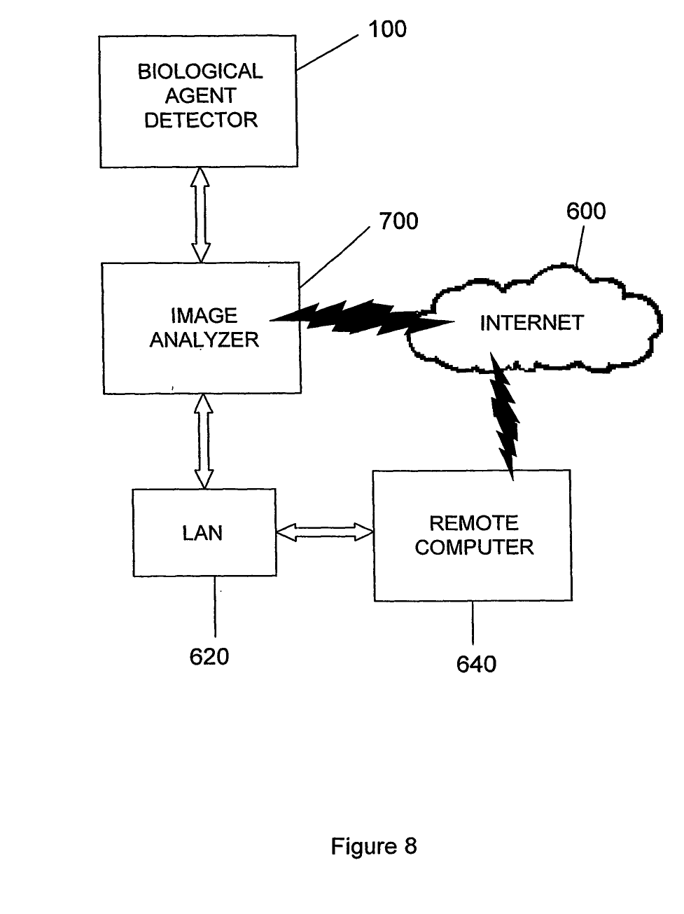

FIG. 8 is a schematic diagram of a biological agent detection system, showing a biological agent detector 100 operatively linked to an image analyzer 700. The image analyzer 700 may be separate, but is preferably integrated into the same housing with the biological agent detector 100. The image analyzer 700 is operatively connected via communication links to a local area network (LAN) 620, the Internet 600 and remote computer system 640. Operative communication links can be wired or wireless. In preferred embodiments, communications between image analyzer 700 and a local area network (LAN) 620, the Internet 600 and remote computer system 640 conform to relevant industry standards such as Health Level Seven (HL7), IEEE1073 (ISO 11073) and IEEE 802.

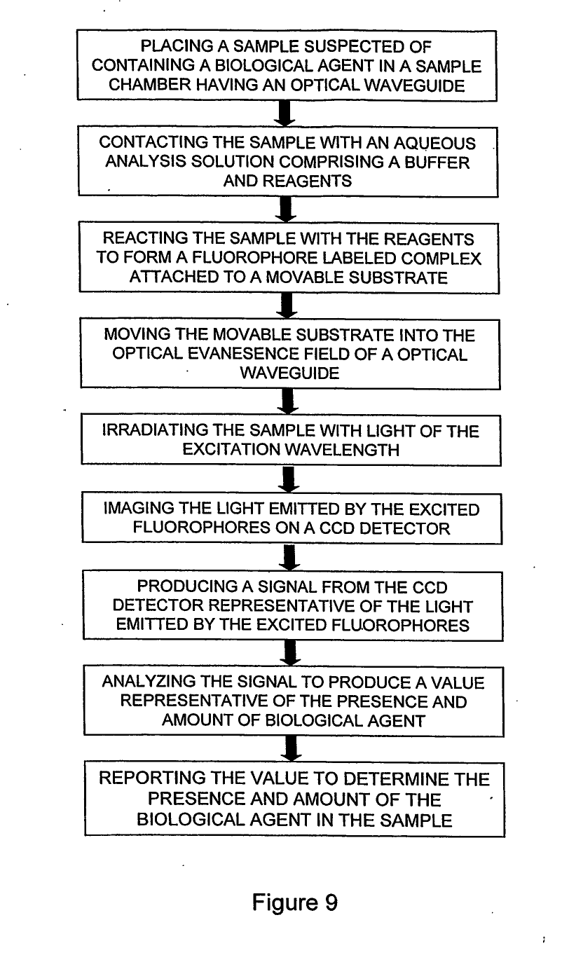

One preferred embodiment of the method of the present invention is illustrated in FIG. 9. The method comprises the steps of placing a sample suspected of containing a biological agent in a sample chamber; contacting the sample with an aqueous analysis solution comprising a buffer and reagents including a first antibody fixed to a movable substrate, first antibody molecules conjugated to a first fluorophore in solution, a second antibody that is conjugated to a second fluorophore having an emission spectrum distinguishable from that of the first fluorophore, wherein the first antibody is specific to the biological agent and the second antibody is specific for an antigen that is irrelevant to the biological agent; reacting the sample with the reagents to form a complex labeled by the first fluorophore attached to the movable substrate; moving the movable substrate into the optical evanescence field of the optical waveguide using a magnetic field; irradiating the sample with light of the excitation wavelength of the first fluorophore and the second fluorophore; imaging the light emitted by all excited fluorophores on a detector, producing a signal representative of the light emitted by all excited fluorophores, analyzing the signal to produce a value representative of the presence and amount of the biological agent based on the specific binding of the first antibody; and reporting the value to determine the presence and amount of the biological agent in a sample.

The claims should not be read as limited to the described order or elements unless stated to that effect. Therefore, all embodiments that come within the scope and spirit of the following claims and equivalents thereto are claimed as the invention.

What is claimed:

1. A biological agent detector comprising:
a sample chamber having an optical waveguide and containing an analysis solution comprising a first antibody specific to the biological agent wherein the first antibody is affixed to a substrate that undergoes relative movement with respect to the analysis solution in response to applied external force,
a first complex of the first antibody and a first fluorophore;
an excitation light source;
an optical system that projects an image of light emitted by excited molecules of the first fluorophore onto a photodetector array wherein the photodetector array produces an output signal that is representative of the position, intensity and wavelength of the light emitted by excited molecules of the first fluorophore; and
an image analyzer that processes the electronic signal representation of the image produced by the photodetector array.

2. The biological agent detector of claim 1 wherein the analysis solution further comprises a second soluble complex of a second antibody and a second fluorophore, wherein the second fluorophore is distinguishable from the first fluorophore by spectral characteristics.

3. The biological agent detector of claim 1 wherein the biological agent is selected from the group consisting of pathogenic microorganisms and biological toxins.

4. The biological agent detector of claim 1 wherein the biological agent is selected from the group consisting of Yersinia pestis (plague), variola major (smallpox), Bacillus anthracis (anthrax), Francisella tularensis (tularaemia), filoviruses such as Ebola hemorrhagic fever and Marburg hemorrhagic fever, arenaviruses such as Lassa (Lassa fever), Junin (Argentine hemorrhagic fever) and related viruses, alphaviruses (Venezuelan encephalomyelitis, eastern & western equine encephalomyelitis), Coxiella burnetti (Q fever), Brucella species (brucellosis), Burkholderia mallei (glanders), Salmonella species, Shigella dysenteriae, Escherichia coli O157:H7, Vibrio cholerae, Cryptosporidium parvum, nipah virus, hantaviruses, tickborne hemorrhagic fever viruses, tickborne encephalitis viruses, yellow fever, multidrug-resistant tuberculosis, Clostridium botulinum toxin (botulism), ricin toxin from Ricinus communis (castor beans), epsilon toxin of Clostridium perfringens, Staphylococcus enterotoxin B and mixtures thereof.

5. The biological agent detector of claim 1 wherein the analysis solution is an aqueous solution further comprising a buffer.

6. The biological agent detector of claim 1 wherein the first antibody is selected from the group consisting of a polyclonal antibody specific for the biological agent; a monoclonal antibody specific for the biological agent; an antibody fragment specific for the biological agent; a recombinant antibody specific for the biological agent and mixtures thereof.

7. The biological agent detector of claim 1 wherein the second antibody is selected from the group consisting of a polyclonal antibody specific for an irrelevant protein; a monoclonal antibody specific for an irrelevant protein; an antibody fragment specific for an irrelevant protein; a recombinant antibody specific for an irrelevant protein; and mixtures thereof.

8. The biological agent detector of claim 7 wherein the analysis solution further comprises a third antibody specific for a second biological agent.

9. The biological agent detector of claim 8 wherein the analysis solution further comprises a fourth antibody specific for a third biological agent.

10. The biological agent detector of claim 9 wherein the analysis solution further comprises a fifth antibody specific for a fourth biological agent.

11. The biological agent detector of claim 1 wherein the substrate is a paramagnetic bead.

12. The biological agent detector of claim 1 further comprising an actuator that provides an external force effective to sweep the substrate through the analysis solution.

13. The biological agent detector of claim 12 wherein the actuator is a permanent magnet.

14. The biological agent detector of claim 12 wherein the actuator is an electromechanical device.

15. The biological agent detector of claim 1 wherein the optical waveguide is movably disposed within the sample chamber.

16. The biological agent detector of claim 15 wherein the first antibody is affixed to the optical waveguide.

17. The biological agent detector of claim 1 wherein the excitation light source is selected from the group consisting of xenon arc lamp, xenon flash lamp, light emitting diode (LED), laser diode or laser.

18. The biological agent detector of claim 1 further comprising a second excitation light source selected from the group consisting of xenon arc lamp, xenon flash lamp, light emitting diode (LED), laser diode or laser.

19. The biological agent detector of claim 1 wherein the photodetector array is a charge coupled device detector.

20. The biological agent detector of claim 2 wherein the second fluorophore is distinguishable from the first fluorophore by characteristics of the emission spectra.

21. The biological agent detector of claim 2 wherein the second fluorophore is distinguishable from the first fluorophore by characteristics of the excitation spectra.

22. The biological agent detector of claim 21 wherein the first fluorophore is excited by a first excitation light source and the second fluorophore is excited by a second excitation light source.

23. The biological agent detector of claim 2 wherein the image analyzer compares the electronic signal representation of the image of light emitted by excited molecules of the first fluorophore to the electronic signal representation of the image of light emitted by excited molecules of the second fluorophore.

24. The biological agent detector of claim 1 further comprising a display.

25. The biological agent detector of claim 1 further comprising a keyboard.

26. The biological agent detector of claim 1 further comprising a bar code reader or a modem.

27. The biological agent detector of claim 23 wherein the electronic signal representation of the image of light emitted by excited molecules of the first fluorophore to the electronic signal representation of the image of light emitted by excited molecules of the second fluorophore are compared ratiometrically.

28. A biological agent detector comprising:
a sample chamber having an optical waveguide and containing an analysis solution comprising a first antibody specific to the biological agent wherein the first antibody is affixed to a substrate; a first complex of the first antibody and a first fluorophore;
a second complex of a second antibody and a second fluorophore, wherein the second fluorophore is distinguishable from the first fluorophore by spectral characteristics
an excitation light source;
a photodetector array optically coupled to the sample chamber; wherein the photodetector array produces an output signal that is representative of the position, intensity and wavelength of the light emitted by excited molecules of the first fluorophore; and
an image analyzer that processes the electronic signal representation of the image produced by the photodetector array wherein the image analyzer compares the electronic signal representation of the image of light emitted by excited molecules of the first fluorophore to the electronic signal representation of the image of light emitted by excited molecules of the second fluorophore.

29. The biological agent detector of claim 28 wherein the analysis solution further comprises a second soluble complex of the second antibody and a second fluorophore, wherein the second fluorophore is distinguishable from the first fluorophore by spectral characteristics.

30. The biological agent detector of claim 28 wherein the biological agent is selected from the group consisting of pathogenic microorganisms and biological toxins.

31. The biological agent detector of claim 28 wherein the biological agent is selected from the group consisting of Yersinia pestis (plague), variola major (smallpox), Bacillus anthracis (anthrax), Francisella tularensis (tularaemia), filoviruses such as Ebola hemorrhagic fever and Marburg hemorrhagic fever, arenaviruses such as Lassa (Lassa fever), Junin (Argentine hemorrhagic fever) and related viruses, alphaviruses (Venezuelan encephalomyelitis, eastern & western equine encephalomyelitis), Coxiella burnetti (Q fever), Brucella species (brucellosis), Burkholderia mallei (glanders), Salmonella species, Shigella dysenteriae, Escherichia coli O157:H7, Vibrio cholerae, Cryptosporidium parvum, nipah virus, hantaviruses, tickborne hemorrhagic fever viruses, tickborne encephalitis viruses, yellow fever, multidrug-resistant tuberculosis, Clostridium botulinum toxin (botulism), ricin toxin from Ricinus communis (castor beans), epsilon toxin of Clostridium perfringens, Staphylococcus enterotoxin B and mixtures thereof.

32. The biological agent detector of claim 28 wherein the analysis solution is an aqueous solution further comprising a buffer.

33. The biological agent detector of claim 28 wherein the first antibody is selected from the group consisting of a polyclonal antibody specific for the biological agent; a monoclonal antibody specific for the biological agent; an antibody fragment specific for the biological agent, a recombinant antibody specific for the biological agent and mixtures thereof.

34. The biological agent detector of claim 29 wherein the second antibody is selected from the group consisting of a polyclonal antibody specific for an irrelevant protein; a monoclonal antibody specific for an irrelevant protein; an antibody fragment specific for an irrelevant protein; a recombinant antibody specific for an irrelevant protein; and mixtures thereof.

35. The biological agent detector of claim 29 wherein the analysis solution further comprises a third antibody specific for a second biological agent.

36. The biological agent detector of claim 35 wherein the analysis solution further comprises a fourth antibody specific for a third biological agent.

37. The biological agent detector of claim 36 wherein the analysis solution further comprises a fifth antibody specific for a fourth biological agent.

38. The biological agent detector of claim 28 wherein the first antibody is affixed to a paramagnetic bead.

39. The biological agent detector of claim 28 further comprising an actuator that provides an external force effective to sweep the substrate through the analysis solution.

40. The biological agent detector of claim 28 wherein the actuator is a permanent magnet.

41. The biological agent detector of claim 28 wherein the optical waveguide is movably disposed within the sample chamber.

42. The biological agent detector of claim 41 wherein the first antibody is affixed to the optical waveguide.

43. The biological agent detector of claim 39 wherein the actuator is an electromechanical device.

44. The biological agent detector of claim 28 wherein the excitation light source is selected from the group consisting of xenon arc lamp, xenon flash lamp, light emitting diode (LED), laser diode or laser.

45. The biological agent detector of claim 28 further comprising a second excitation light source selected from the group consisting of xenon arc lamp, xenon flash lamp, light emitting diode (LED), laser diode or laser.

46. The biological agent detector of claim 28 wherein the photodetector is a charge coupled device detector.

47. The biological agent detector of claim 29 wherein the second fluorophore is distinguishable from the first fluorophore by characteristics of the emission spectra.

48. The biological agent detector of claim 29 wherein the second fluorophore is distinguishable from the first fluorophore by characteristics of the excitation spectra.

49. The biological agent detector of claim 29 wherein the first fluorophore is excited by a first excitation light source and the second fluorophore is excited by a second excitation light source.

50. The biological agent detector of claim 28 further comprising an image analyzer.

51. The biological agent detector of claim 28 further comprising a processor.

52. The biological agent detector of claim 28 further comprising a display.

53. The biological agent detector of claim 28 further comprising keyboard.

54. The biological agent detector of claim 28 further comprising a bar code reader.

55. The biological agent detector of claim 28 further comprising a modem.

56. The biological agent detector of claim 28 wherein the detector is adapted for handheld use by an operator in protective clothing.

57. A method for determining the presence and amount of a biological agent in a sample comprising the steps of:
placing the sample in a sample chamber having an optical waveguide;
contacting the sample with an analysis solution comprising a buffer and reagents, the reagents comprising a first antibody affixed to a substrate, and a conjugate of the first antibody and a first fluorophore, wherein the first antibody is specific to the biological agent comprising a target analyte;
reacting the sample with the reagents to form a complex of the target analyte with the first antibody labeled by the first fluorophore;
moving the complex into the optical evanescence field of a optical waveguide;
irradiating the optical evanescence field with an excitation light source;
imaging the light emitted by excited fluorophores on a photodetector array;
producing a signal representative of the light emitted by excited fluorophores;
processing the signal to produce a value representative of the presence and amount of the biological agent based on the specific binding of the first antibody; and
reporting the value to determine the presence and amount of the biological agent in a sample.

58. The method of claim 57 wherein the analysis solution further comprises a second antibody that is conjugated to a second fluorophore that is distinguishable from the first fluorophore by spectral characteristics, wherein the second antibody is specific for an antigen that is irrelevant to the biological agent.

59. The method of claim 57 wherein the biological agent is selected from the group consisting of pathogenic microorganisms and biological toxins.

60. The method of claim 57 wherein the biological agent is selected from the group consisting of Yersinia pestis (plague), variola major (smallpox), Bacillus anthracis (anthrax), Francisella tularensis (tularaemia), filoviruses such as Ebola hemorrhagic fever and Marburg hemorrhagic fever, arenaviruses such as Lassa (Lassa fever), Junin (Argentine hemorrhagic fever) and related viruses, alphaviruses (Venezuelan encephalomyelitis, eastern & western equine encephalomyelitis), Coxiella burnetti (Q fever), Brucella species (brucellosis), Burkholderia mallei (glanders), Salmonella species, Shigella dysenteriae, Escherichia coli O157:H7, Vibrio cholerae, Cryptosporidium parvum, nipah virus, hantaviruses, tickborne hemorrhagic fever viruses, tickborne encephalitis viruses, yellow fever, multidrug-resistant tuberculosis, Clostridium botulinum toxin (botulism), ricin toxin from Ricinus commune (castor beans), epsilon toxin of Clostridium perfringens, Staphylococcus enterotoxin B and mixtures thereof.

61. The method of claim 57 wherein the analysis solution is an aqueous solution further comprising a buffer.

62. The method of claim 57 wherein the first antibody is selected from the group consisting of a polyclonal antibody specific for the biological agent; a monoclonal antibody specific for the biological agent; an antibody fragment specific for the biological agent; a recombinant antibody specific for the biological agent and mixtures thereof.

63. The method of claim 58 wherein the second antibody is selected from the group consisting of a polyclonal antibody specific for an irrelevant protein; a monoclonal antibody specific for an irrelevant protein; an antibody fragment specific for an irrelevant protein; a recombinant antibody specific for an irrelevant protein; and mixtures thereof.

64. The method of claim 63 wherein the aqueous analysis solution further comprises a third antibody specific for a second biological agent.

65. The method of claim 64 wherein the aqueous analysis solution further comprises a fourth antibody specific for a third biological agent.

66. The method of claim 65 wherein the aqueous analysis solution further comprises a fifth antibody specific for a fours biological agent.

67. The method of claim 57 wherein the substrate is a paramagnetic bead.

68. The method of claim 57 further comprising the step of using an actuator to sweep the substrate through the analysis solution.

69. The method of claim 68 wherein the actuator is a permanent magnet.

70. The method of claim 57 wherein the optical waveguide is movably disposed within the sample chamber.

71. The method of claim 57 wherein the substrate is an optical waveguide.

72. The method of claim 68 wherein the actuator is an electromechanical device.

73. The method of claim 57 wherein the excitation light source is selected from the group consisting of xenon arc lamp, xenon flash lamp, light emitting diode (LED), laser diode or laser.

74. The method of claim 57 further comprising a second excitation light source selected from the group consisting of xenon arc lamp, xenon flash lamp, light emitting diode (LED), laser diode or laser.

75. The method of claim 57 wherein the photodetector array is a charge coupled device detector.

76. The method of claim 58 wherein the second fluorophore is distinguishable from the first fluorophore by characteristics of the emission spectra.

77. The method of claim 58 wherein the second fluorophore is distinguishable from the first fluorophore by characteristics of the excitation spectra.

78. The method of claim 77 wherein the first fluorophore is excited by a first excitation light source and the second fluorophore is excited by a second excitation light source.
